(19) United States
(12) Patent Application Publication
(10) Pub. No.: US 2009/0197800 A1
(43) Pub. Date: Aug. 6, 2009

(54) Insulin Receptor Binding Peptides with Non-Insulin Gene Activation Profiles and Uses Thereof

(75) Inventors: Lauge Schaffer, Copenhagen O (DK); Klaus Stensgaard Frederiksen, Frederiksberg (DK)

Correspondence Address: NOVO NORDISK, INC.;INTELLECTUAL PROPERTY DEPARTMENT, 100 COLLEGE ROAD WEST, PRINCETON, NJ, 08540, US

(73) Assignee: Novo Nordisk A/S, Bagsvaerd (DK)

(21) Appl. No.: 11/718,159

(22) PCT Filed: Oct. 14, 2005
(86) PCT No.: PCT/EP05/55264

§ 371 Date: Apr. 16, 2009

Related U.S. Application Data
(60) Provisional application No. 60/622,588, filed on Oct. 27, 2004.

(51) Int. Cl. A61K 38/30; A61P 3/10

(52) U.S. Cl. 514/12

(57) ABSTRACT

Methods for binding insulin receptors (and typically activating one or more function of an insulin receptor) by contacting insulin receptor-presenting cells, such as cells in a subject, with an effective amount of one or more insulin receptor binding peptides, where upregulation of one or more components of the insulin receptor-associated cholesterol synthesis pathway is not desired, are provided.

### FIELD OF THE INVENTION

The invention described here pertains to insulin receptor (IR) binding peptides (IRBPs) having gene activation profiles that differ from human insulin, compositions comprising such peptides, and methods of using such peptides and compositions.

### BACKGROUND OF THE INVENTION

Insulin is a potent metabolic and growth promoting hormone that acts on cells to stimulate glucose, protein, and lipid metabolism, as well as RNA and DNA synthesis. A well-known effect of insulin is the regulation of glucose levels in the body. This effect occurs predominantly in liver, fat, and muscle tissue. In the liver, insulin stimulates glucose incorporation into glycogen and inhibits the production of glucose. In muscle and fat tissue, insulin stimulates glucose uptake, storage, and metabolism. Defects in glucose utilization are very common in the population, giving rise to diabetes.

Insulin initiates signal transduction in target cells by binding to a cell surface insulin receptor (IR). The human IR is a glycoprotein having molecular weight of 350-400 kDa (depending of the level of glycosylation). It is synthesized as a single polypeptide chain and proteolytically cleaved to yield a disulfide-linked α-β insulin monomer. Two α-β monomers are linked by disulfide bonds between the α-subunits to form a dimeric form of the receptor (β-α-α-β-type configuration). A human IR α-subunit typically is comprised of 723 amino acids, and it can be divided into two large homologous domains, L1 (amino acids 1-155) and L2 (amino acids 313-468), separated by a cysteine rich region (amino acids 156-312) (Ward et al., 1995, Prot. Struct. Funct Genet. 22:141-153). Many determinants of insulin binding seem to reside in the α-subunit of the human IR. The human IR appears to be in dimeric form in the absence of ligand.

A binding model for IRs such as the human IR has been presented. This model proposes an IR comprising two insulin binding sites positioned on two different surfaces of the receptor molecule, such that each alpha-subunit is involved in insulin binding. In this way, activation of the insulin receptor is believed to involve cross-connection of the α-subunits by insulin.

### BRIEF SUMMARY OF THE INVENTION

The invention described here provides a method of binding, and typically of activating at least one function of, an insulin receptor, on an insulin receptor presenting cell, typically in a mammal, such as a human, wherein upregulation of one or more components of the insulin receptor (IR)-associated cholesterol synthesis (IRACS) pathway is undesirable, by delivering to the cell an effective amount of an insulin receptor binding peptide (IRBP—as defined further herein).

In a particular exemplary aspect, the invention provides a method of reducing blood glucose level in a subject having a condition in which upregulation of the IRACS pathway is undesirable comprising delivering to the subject a physiologically effective amount of an IRBP so as to reduce blood level therein. The subject typically is a human patient that has diabetes or pre-diabetes. Commonly, the subject also has at least one additional high cholesterol condition (HCC)-associated heart disease risk factors (HHDRFs) or a known HCC. In a specific facet, the method is practiced upon a human patient having a total cholesterol level of more than about 200 mg/dl and/or a total LDL cholesterol level of more than about 100 mg/dl (e.g., a human patient that frequently has a total cholesterol level of more than about 230 mg/dl and/or a total LDL cholesterol level of more than about 130 mg/dl). IRBPs can be delivered by any suitable method (e.g., by direct administration or expression from a suitable nucleic acid which may be comprised in a recombinant host cell or vector for delivery). In a particular aspect, one or more IRBPs are delivered by pulmonary administration. In another particular aspect, one or more IRBPs are delivered by oral administration. In additional aspects, IRBPs are also or alternatively administered with (a) one or more secondary anti-diabetic agents and/or (b) one or more anti-HCC/anti-HHDRF agents. In an additional facet, the invention provides such methods wherein an approximately equivalent amount of human insulin upregulates expression of HMG-CoA reductase by at least two times the level expressed upon delivery of the IRBP to the subject. In a further aspect, the invention relates to the use of an IRBP in the manufacture of a medicament used in the treatment of diabetes or pre-diabetes in a patient having a condition that renders upregulation of the IRACS pathway undesirable.

These and other advantageous aspects of the invention are further described elsewhere herein.

### BRIEF DESCRIPTION OF THE FIGURES

FIG. 1 shows HMG-CoA reductase mRNA expression levels in SGBS adipocytes treated with increasing concentrations of human insulin (INS) or IRBP S597. All expression levels were normalized to 18S expression levels.

FIG. 2 shows quantitative RT-PCR data for HMG-CoA synthase 1 and mevalonate (diphospho) decarboxylase expression upon delivery of an equivalent amount of an IRBP (S597) or human insulin (INS) to SGBS-adipocytes. All expression levels were normalized to 18S expression levels.

FIG. 3 shows levels of HMG-CoA reductase, HMG-CoA synthase 1, and mevalonate (diphospho) decarboxylase mRNA expression in primary rat hepatocytes treated with increasing concentrations of human insulin (INS) or IRBP S597. All expression levels were normalized to 18S expression levels.

FIG. 4 shows Glucose-6-phosphate catalytic subunit and fatty acid synthase mRNA expression levels in primary rat hepatocytes treated with increasing concentrations of human insulin (INS) or IRBP S597. All expression levels were normalized to 18S expression levels.

### DETAILED DESCRIPTION OF THE INVENTION

The invention described here provides various methods of modulating physiological responses, diagnosing conditions, etc., which methods relate to binding of an insulin receptor (IR) by an insulin receptor binding protein (IRBP) (as defined below) in subjects where upregulation of cholesterol biosynthesis is considered undesirable (e.g., a human patient suffering from one or more ailments related to a high cholesterol condition).

In a particularly useful aspect, the invention provides a method of increasing or enhancing one or more insulin receptor signaling activities, such as lowering of blood glucose, in a subject having a condition wherein upregulation of IR activation-associated cholesterol synthesis (IRACS) is undesirable, comprising delivering to the subject an effective amount of an IRBP under conditions such that the IRACS pathway is upregulated substantially less than it would be with the use of insulin in place of the IRBP. In an even more specific aspect, the invention provides such methods wherein one or more components of the IRACS pathway are not upregulated.

In even more particular facet, the invention relates to the use of an IRBP to treat a disease, disorder, or condition wherein upregulation of one or more aspects of IR signaling associated with IRBP-IR interactions is deemed beneficial (e.g., lowering of blood glucose levels) without upregulation of IRACS. In a further aspect, the invention relates to the use of an IRBP, an IRBP-containing composition, or related molecule/composition (e.g., a nucleic acid molecule comprising a sequence encoding an IRBP or a cell, vector, or composition comprising such a nucleic acid molecule) for the preparation of a medicament for treating a disease, disorder, or condition wherein upregulation of IRBP-IR-associated signaling (IRBPIRAS) is desirable but wherein upregulation of IRACS is undesirable (e.g., for treating diabetes in a patient having an unhealthy high cholesterol condition (HCC)).

A “therapeutically effective amount” refers to an amount of a biologically active compound or composition that, when delivered in appropriate dosages and for appropriate periods of time to a host that typically is responsive for the compound or composition, is sufficient to achieve a desired therapeutic result in a host and/or typically able to achieve such a therapeutic result in substantially similar hosts (e.g., patients having similar characteristics as a patient to be treated). A therapeutically effective amount of an IRBP may vary according to factors such as the disease state, age, sex, and weight of the individual, and the ability of the IRBP to elicit a desired response in the individual. A therapeutically effective amount is also one in which any toxic or detrimental effects of the agent are outweighed by the therapeutically beneficial effects. Exemplary therapeutic effects include, e.g., (a) a reduction in the severity of a disease, disorder, or related condition in a particular subject or a population of substantial similar subject; (b) a reduction in one or more symptoms or physiological conditions associated with a disease, disorder, or condition; and/or (c) a prophylactic effect. A reduction of the severity of a disease can include, for example, (a) a measurable reduction in the spread of a disorder; (b) an increase in the chance of a positive outcome in a subject (e.g., an increase of at least about 5%, 10%, 15%, 20%, 25%, or more); (c) an increased chance of survival or lifespan; and/or (d) a measurable reduction in one or more biomarkers associated with the presence of the disease state (e.g., a reduction in the amount and/or severity of diabetic symptoms; etc.). A therapeutically effective amount can be measured in the context of an individual subject or, more commonly, in the context of a population of substantial similar subjects (e.g., a number of human patients with a similar disorder enrolled in a clinical trial involving a IRBP composition or a number of non-human mammals having a similar set of characteristics being used to test a IRBP in the context of preclinical experiments).

IRBPs also can be delivered to a host in a prophylactically effective amount as part of a disease/disorder prevention program or for otherwise increasing general health. A “prophylactically effective amount” refers to an amount of an active compound or composition that is effective, at dosages and for periods of time necessary, in a host typically responsive to such compound or composition, to achieve a desired prophylactic result in a host or typically able to achieve such results in substantially similar hosts. Exemplary prophylactic effects include a reduction in the likelihood of developing a disorder, a reduction in the intensity or spread of a disorder, an increase in the likelihood of survival during an imminent disorder, a delay in the onset of a disease condition, a decrease in the spread of an imminent condition as compared to in similar patients not receiving the prophylactic regimen, etc. Typically, because a prophylactic dose is used in subjects prior to or at an earlier stage of disease, the prophylactically effective amount will be less than the therapeutically effective amount for a particular IRBP. A prophylactic effect also can include, e.g., a prevention of the onset, a delay in the time to onset, a reduction in the consequent severity of the disease as compared to a substantially similar subject not receiving IRBP composition, etc.

IRBPs can be delivered to a host or cells in a physiologically effective amount. A physiologically effective amount is an amount of an active agent that upon administration to a host that is normally responsive to such an agent results in the induction, promotion, and/or enhancement of at least one physiological effect associated with modulation of IR activity (e.g., modulation of IR phosphorylation, reduction in blood glucose levels, and/or IR-associated signaling).

“Treatment” generally refers to the delivery of an effective amount of a therapeutically active compound with the purpose of preventing any symptoms of disease or disease state (or underlying conditions of a disease) to develop or with the purpose of easing, ameliorating, or eradicating (curing) such symptoms or disease states already developed. The term “treatment” is thus meant to include prophylactic treatment. However, it will be understood that therapeutic regimens and prophylactic regimens of the invention also can be considered separate and independent aspects of this invention.

To better illustrate the invention, a discussion of particular target cells, tissues, subjects, patients, etc. of these and other methods and uses provided by the invention is provided below followed by a description of IRBPs.

### A. Cells and Subjects

At least one aspect of this invention is embodied in the discovery that effective amounts of one or more IRBPs can be provided to cells (e.g., in vitro, ex vivo, or in the cells of a subject in vivo) that display insulin receptors with the effect of binding thereto (which may be relevant in, e.g., delivery of other agents to IR displaying cells and/or for diagnostic purposes), and typically with the effect of further causing at least partial activation thereof, without upregulating one or more aspects of the cell's IRACS pathway (e.g., without upregulating IR activation-associated HMG-CoA reductase expression).

In another particular aspect, the invention provides a method of modulating IR signaling in a patient comprising delivering one or more IRBPs to a patient having a disease, disorder, or condition wherein activation of insulin receptor signaling is considered beneficial, such as diabetes or an insulin resistance condition, but wherein upregulation of the IRACS pathway is considered detrimental.

Unless otherwise stated, subjects in the context of various inventive methods described herein are vertebrates, e.g., chordates, typically mammals (such as livestock, household pets, test rats, dogs, guinea pigs, mice, hamsters, pigs, primates, etc.), and most commonly human patients, having or being at substantial risk of developing (and typically of soon developing) at least one condition in which upregulation of an IRACS pathway is undesirable or even detrimental.

A substantial risk of developing a condition typically means that there is some substantial basis in the physiological state, environment, and/or genetic characteristics of the applicable subject that indicate that a condition will likely develop in the subject in which upregulation of the IRACS pathway will be considered undesirable or detrimental. Typically, a substantial risk of developing such a condition means that a person of ordinary skill in the relevant field would consider it a very real possibility (if not likely) that the relevant condition(s) will soon develop (e.g., within a period of a few years or less) unless medical intervention, lifestyle changes, and/or other steps are taken that eliminate such risk(s). The likelihood of developing a condition will vary with the relevant conditions and such factors. Although generalizations are difficult to make given the various uses of IRBPs, a substantial risk may mean a risk of about 20% or great, about 25% or greater, about 30% or greater, about 35% or greater, about 40% or greater, about 50% or greater, about 60% or greater, about 70% or greater, about 75% or greater, about 80% or greater, about 85% or greater, about 90% or greater, or about 95-99% of developing the condition(s) (e.g., as assessed by diagnosis of a qualified healthcare professional and/or application of models based on similar patients/subjects).

In one aspect, IRBPs can be delivered to a subject (e.g., a human patient, a household pet, or other mammal such as a laboratory test animal or livestock) (a) having a diagnosis and/or physiological conditions indicative of a state that suggest upregulation of the IRACS pathway is undesirable and (b) suffering from a form of idiopathic diabetes mellitus, such as Type 1 insulin dependent diabetes mellitus (IDDM) or Type 2 IDDM (e.g., a patient having a fasting plasma glucose level or about or in excess of about 126 mg/dL (7 mmol/L) and/or having plasma glucose levels of about or in excess of about 200 mg/dL (11 mmol/L)), typically at two times points during a glucose tolerance test (GTT), one of which is taken typically within 2 hrs of ingestion of glucose). In one aspect, the subject is a human patient having the conditions of and/or a diagnosis of late onset Type 2 diabetes associated with obesity.

In a further facet, methods of the invention may be practiced to reduce the risk of developing a disease condition in a subject that has conditions associated with and/or that is diagnosed as having a pre-diabetes condition and a condition in which upregulation of the IR-associated cholesterol pathway is detrimental and/or undesirable (e.g., a patient having a fasting blood glucose level that is at about or is above about 100 mg/dL, but less than about 125 mg/dL, and whose glucose levels are at least about 140 mg/dL but less than about 200 mg/dL following an oral glucose tolerance test (OGTT)). In an additional facet, a physiologically effective amount or prophylactically effective amount of an IRBP is delivered to a patient that is obese and suffers from an endocrine autoimmunity condition that is often associated with IDDM (e.g., Addison disease) and/or that has a family member that is diagnosed as having IDDM. One or more IRBPs also or alternatively can be provided in such amounts to subjects that possess islet cell cytoplasmic antibodies (ICCAs) suggestive of a pre-diabetes state so as to reduce the risk of developing or further developing a diabetic condition. One or more IRBPs can be similarly also or alternatively provided in such amounts to subjects that possess islet cell surface antibodies (ICSAs) in amounts suggestive of a diabetes or pre-diabetes condition so as to reduce the risk of developing or further developing a diabetic condition. An IRBP also or alternatively can be administered in either such amount to subjects having antibodies to glutamic acid decarboxylase (GAD), optionally with the presence of one or more risk indicators for development of diabetes (e.g., obesity, a family member with IDDM, etc.), so as to reduce the risk of developing or further developing a diabetic condition. In a further aspect, an IRBP also or alternatively can be provided to a subject having anti-insulin antibodies (IM) suggestive of diabetes or pre-diabetes, so as to reduce the risk of developing or further developing a diabetic condition. In another facet, an IRBP also or alternatively can be delivered in either such amounts to a subject also or alternatively having a significant loss of pancreatic β cells and/or abnormal functioning of pancreatic a cells (and optionally one or more further risk factors for developing or having diabetes), so as to reduce the risk of developing or further developing a diabetic condition. In an additional aspect, an IRBP also or alternatively can be delivered in either such amounts to a subject also or alternatively having hyperglycemia and abnormally high levels of glucagon secretion (optionally with one or more further diabetes development risk factors), so as to reduce the risk of developing or further developing a diabetic condition. In a further facet, an IRBP also or alternatively can be delivered in either such amounts to a subject also or alternatively exhibiting ketoacidosis (optionally with one or more further diabetes development risk factors), so as to reduce the risk of developing or further developing a diabetic condition. In a further facet, an IRBP also or alternatively can be delivered in either such amounts to a subject that also or alternatively exhibits a reduced ability to secrete glucagon in response to hypoglycemia (optionally with one or more further diabetes development risk factors), so as to reduce the risk of developing or further developing a diabetic condition. In yet another aspect, an IRBP also or alternatively can be delivered in either such amounts to a subject that also or alternatively exhibits a hemoglobin A1c (HbA1c) of about 6% or more (e.g., about 6.5-9%), for example about 7% or higher (e.g., about 8% or higher, such as about 9% or higher), optionally with one or more further diabetes development risk factors, so as to reduce the risk of developing or further developing a diabetic condition.

In particular aspects, the invention relates to a method of reducing the risk of developing or further developing a condition associated with a diabetic or metabolic syndrome state, such as a form of microvascular disease or condition associated with a microvascular disease (e.g., retinopathy, nephropathy, proteinuria (e.g., microalbuminuria), and neuropathy) or a form of a macrovascular disease (such as coronary artery disease (CAD), cerebrovascular disease, and peripheral vascular disease (PVD)). The risk of developing such conditions may be reduced by about 20% or more, about 30% or more, about 40% or more, about 50% or more, or even about 60% or more by practice of various methods provided here.

In another facet, the invention provides a method of improving metabolic control in a subject having an IR-associated metabolic control disorder and a condition and/or diagnosis suggesting that the subject has or is at risk of developing a cardiovascular disorder comprising delivering an effective amount of one or more IRBPs to the subject under conditions such that at least one component of the IRACS pathway is not upregulated.

In still another aspect, the invention relates to methods of regulating metabolism in a subject having a condition in which upregulation of the IRACS pathway is undesirable or detrimental, comprising delivering an effective amount of one or more IRBPs to the subject, wherein practice of the method causes a reduction in the risk of heart disease, delays the onset of heart disease, and/or reduces the risk of heart disease-associated fatality.

In methods described herein, therapeutic and prophylactic effects (e.g., various endpoints) can be assessed either with respect to the individual subject, a population of similar subjects (e.g., a class of patients enrolled in a clinical trial), or both.

In another aspect, one or more IRBPs are provided in physiologically effective, prophylactically effective, and/or therapeutically effective amounts to a subject having conditions indicative of diabetes or pre-diabetes, which conditions include one or more of elevated levels of free fatty acids in the plasma, uncontrolled lipolysis in adipose tissue, suppressed glucose metabolism in peripheral tissues (e.g., skeletal muscle), poor glucose utilization in peripheral tissues (e.g., adipose tissues and/or skeletal muscle), abnormally increased hepatic glucose output, abnormally low malonyl-CoA levels, abnormally high transport of fatty acyl-CoAs to the mitochondria, hypertriglyceridemia, increased catabolism of protein and/or abnormally high levels of plasma amino acids, increased hepatic triglyceride production, dyslipidemia, abnormally high levels of very low density lipoproteins (VLDLs) in the circulation, decreased expression of one or more genes necessary for target tissues to respond normally to insulin (e.g., glucokinase in liver, the GLUT 4 class of glucose transporters in adipose tissue, or both).

In an additional aspect, the invention provides a method of preventing, reducing the risk of developing, or treating one or more aspects of metabolic syndrome (syndrome X) or negative health conditions (including or not including diabetes) by delivering to a subject a prophylactically effective and/or therapeutically effective amount of an IRBP so as to prevent, reduce the risk of developing, or treat the aspects of metabolic syndrome. Aspects of metabolic syndrome include visceral adiposity (obesity), insulin resistance, low levels of HDLs, a systemic proinflammatory state, hypertension, dyslipidemia, insulin resistance, chronic inflammation, impaired fibrinolysis, and/or procoagulation. For example, such a method may be used as a treatment for cardiovascular, coagulation, and/or fibrinolysis pathologies associated with metabolic syndrome, such as atherosclerosis.

In a further aspect, methods provided here may be practiced in a subject suffering from or at substantial risk of developing a secondary diabetes mellitus condition. Examples of such conditions include maturity onset type diabetes of the young (MODY—e.g., MODY-1, MODY-2, MODY-3, MODY-4, MODY-5, and MODY-X); pancreatic disease-associated diabetes mellitus (e.g., pancreatectomy-associated diabetes, cystic fibrosis-associated diabetes); endocrine disease-associated diabetes (e.g., diabetes arising from or related to a disease associated with over production of one or more counter-regulatory hormones, such as glucagon, epinephrine, and cortisol—e.g., glucagonoma-associated diabetes; pheochromocytoma-associated diabetes; and Cushing-syndrome associated diabetes); drug-induced diabetes (e.g., glucocorticoid-associated diabetes); anti-insulin receptor autoantibody-associated diabetes; insulin gene mutation-associated diabetes; insulin receptor gene mutation-associated diabetes; and gestational diabetes.

In an additional facet, the invention provides a method of treating diabetes or an impaired glucose tolerance condition in a patient in which upregulation of the IR activation-associated cholesterol synthesis pathway is undesirable comprising delivering an effective amount of an IRBP thereto so as to treat the diabetes or impaired glucose tolerance condition. In particular aspects, such a condition in the patient may arise in association with or as a complication of a syndrome such as lipoatrophic diabetes, Wolfram syndrome, Down syndrome, Klinefelter syndrome, Turner syndrome, myotonic dystrophy, muscular dystrophy, Huntington disease, Friedrich ataxia (or other purine nucleotide phosphorylase deficiency), Prader-Willi syndrome, Werner syndrome, and/or Cockayne syndrome.

Various methods provided herein comprising the delivery of effective amounts of one or more IRBPs to a patient may be applied to help improve the physiological condition (health) of a patient having (a) conditions of and/or a diagnosis of diabetes, pre-diabetes, or other condition wherein upregulation of IRBPIRAS, such as upregulation of IR activity related to lowering blood glucose levels, is desirable (e.g., an insulin resistance condition) and (b) one or more conditions that render it desirable to not upregulate the IR related cholesterol synthesis pathway (such as one of the conditions more specifically described below), with the effect of preventing or lessening the chances of developing (e.g., as compared to without delivering the IRBP) one or more negative consequences associated with diabetes or related condition, such as renal failure, blindness, and limb amputations due to circulatory problems, by delivering an effective amount of an IRBP thereto under conditions suitable for preventing or lessening the chances of developing such conditions.

Undesirability of upregulation of the IRACS pathway can be determined by any suitable standard and may simply entail a prior determination that such upregulation is not sought (e.g., in the context of an in vitro screening assay, experiment, or other procedure). Upregulation of the IRACS pathway may be considered undesirable when an increase in cholesterol level in the subject (particularly of low density lipoprotein (LDL) cholesterol), in combination with one or more other health, genetic, and/or environmental factors (other than having diabetes), significantly increases the likelihood (e.g., increases the likelihood by at least about 5%, at least about 10%, at least about 20%, etc.) of developing a non-diabetes-related cholesterol-associated (NDRCA) disorder, condition, or disease, such as a cardiovascular disease (e.g., heart disease). Upregulation of the IRACS pathway may be considered detrimental when it is more likely than not that an increase in cholesterol in the subject will lead to development or further development of a NDRCA disorder, condition, or disease. Where a patient has a diagnosis or condition that specifically indicates an increase in cholesterol is harmful (e.g., the patient is suffering from heart disease), or is currently medicated to reduce the risk of developing or prolong the time before onset of such a condition (e.g., where a patient is taking a prescribed cholesterol lowering medication to prevent heart disease due to one or more factors besides or in addition to the existence of diabetes or a pre-diabetes state), upregulation of the IRACS pathway also may be considered detrimental. Where a patient has diabetes, pre-diabetes, or a related condition and at least one other risk factor for the development of a cholesterol-related disease, disorder, or condition (e.g., a heart disease), upregulation of the IRACS pathway may be considered undesirable.

In another particular exemplary aspect, the invention provides a method of regulating glucose metabolism in a patient having impaired glucose tolerance (e.g., a condition marked by frequent blood glucose levels of about 140-200 mg/dl about 2 hours after glucose ingestion) and a diagnosis or condition that suggests that upregulation of the IRACS pathway is not desirable comprising delivering an effective amount of one or more IRBPs to the patient.

In a further aspect, the invention provides a method of also or alternatively treating one or more disorders such as hyperlipidemia, obesity, and appetite-related syndromes in a patient wherein upregulation of the IRACS pathway is undesirable comprising delivering to the patient an effective amount of one or more IRBPs. The invention additionally provides a prophylactic regimen against high glucose level-related stroke, kidney disease, and/or blindness in a patient wherein upregulation of the IRACS pathway is undesirable comprising delivery of an effective amount of an IRBP thereto. In another aspect, the invention provides a prophylactic regiment against diabetes or a related condition in a patient having hyperinsulinemia and in which upregulation of the IRACS pathway is undesirable comprising delivering an effective amount of an IRBP thereto. In a further facet, the invention provides a method of reducing blood pressure in a patient having a condition that renders upregulation of the IRACS pathway undesirable comprising delivering to the patient an effective amount of an IRBP. In yet another aspect, the invention provides a method of treating or preventing an IR-associated neurodegenerative disease and/or non-diabetes autoimmune disease comprising in a patient in which upregulation of the IRACS pathway is undesirable, comprising delivering to the patient an effective amount of an IRBP. In still another aspect, the invention provides a method of preventing weight gain in a patient in need thereof and that has a condition that renders upregulation of the IRACS pathway unsuitable comprising delivering to the patient an effective amount of an IRBP. In a further facet, the invention provides a method of treating obesity in a patient wherein upregulation of the IRACS pathway is undesirable comprising administering a therapeutically effective amount of an IRBP to the patient so as to treat obesity (by stabilizing and/or reducing the weight of the patient). In still another aspect, the invention provides a method of treating a patient suffering from a disease condition associated with or caused by hypoglycaemia, hypokalaemia, and/or hypophosphataemia and having a condition that renders upregulation of the IRACS pathway undesirable comprising delivering an effective amount of an IRBP to the patient to treat such conditions/symptoms.

In another aspect, the invention provides the use of an IRBP or IRBP composition (such as a combination composition) in the manufacture of a medicament used in the treatment of any of the foregoing conditions.

In one general aspect, the invention provides a method of modulating glucose levels in a patient having a condition that renders upregulation of the IRACS pathway undesirable comprising administering or otherwise delivering to the patient an effective amount of an IRBP. In another general aspect, the invention provides a method of mediating IR activity in a patient having a condition wherein upregulation of the IRACS pathway is undesirable comprising delivering a physiologically effective amount of an IRBP to the patient such that responsive IR on IR-presenting cells is bound in an amount and under conditions sufficient to induce, promote, enhance, and/or otherwise modulate an IR-mediated activity or response.

In yet a further facet, the invention provides a method of modulating nitric oxide production levels; mediating RAS, RAF, MEK, and/or mitogen-activated protein (MAP) kinase pathways; modulating vascular tissue growth and/or smooth muscle cell, monocyte, macrophage, and/or endothelial cell growth and/or migration; stimulating production of plasminogen activator inhibitor type 1 (PAI-1); modulating endothelin production; modulating IR-associated proatherosclerotic pathway biological events; modulating IR-associated inflammation; treating and/or reducing the risk of arterial injury; treating and/or preventing atherosclerosis in a patient having a condition wherein upregulation of the IRACS pathway is undesirable comprising delivering to the patient an effective amount of an IRBP to induce, promote, and/or enhance such events/responses.

Particular additional and nonlimiting examples of classes of conditions in which upregulation of IRACS pathways are undesirable or detrimental are provided in the following subsections.

### 1. High Cholesterol Condition (HCC) Subjects

An upregulation of the IRACS pathway in subjects that have a high cholesterol condition (HCC) is undesirable and typically detrimental. A high cholesterol condition is a condition in which cholesterol levels have reached a state in which development or further development of HCC-related disease or disorders (e.g., atherosclerosis, cardiovascular disease, etc.) is likely. A HCC can be indicated by (a) total cholesterol levels of more than about 200 mg/dl (e.g., at least about 220 mg/dl, such as about 230 mg/dl or more, such as about 240 mg/dl or more), (b) total triglycerides of more than about 200 mg/dl (e.g., at least about 250 mg/dl, at least about 275 mg/dl, at least about 300 mg/dl, at least about 325 mg/dl, at least about 350 mg/dl, at least about 375 mg/dl, at least about 400 mg/dl, at least about 425 mg/dl, etc.), and/or (c) LDL cholesterol levels of more than about 100 mg/dl (e.g., at least about 130 mg/dl, such as at least about 150 mg/dl, such as at least about 160 mg/dl, at least about 170 mg/dl, at least about 190 mg/dl, or more than about 190 mg/dl). Typically, a HCC condition is determined by (a) and/or (c), although presence of high levels of triglycerides also typically is of relevance to the health of subjects to which delivery of IRBPs may be beneficial.

### 2. HCC-Associated Heart Disease Risk Factor (HHDRF) Subjects

In another aspect, IRBPs are delivered to a subject, typically a human patient, having diabetes, pre-diabetes, an insulin sensitivity disorder, or another related disorder for which IRBPIRAS may be beneficial, wherein the subject also has one or more high cholesterol condition-associated heart disease risk factors (HHDRFs). HHDRFs can be any recognized factor that, taken in combination with a HCC, significantly increases the likelihood of heart disease in a subject. Particular examples of HHDRFs include (1) family history of heart disease; (2) regular cigarette smoking (e.g., smoking an average of about 5 cigarettes or more for a period of one year or longer); (3) high blood pressure (chronic prehypertension or hypertension—e.g., frequent or regular blood pressure measurements of about 120+/about 80+ (systolic/diastolic), for example about 130+/about 85+, such as about 140+/about 90+, e.g., about 150+/about 95+, or any similar combination thereof—e.g., blood pressures of about 120-150+/80-95+), (4) obesity, (5) a high fat and/or high carbohydrate diet (e.g., more than about 50%, more than about 60%, more than about 70%, more than about 80%, etc. of either or a combination thereof), (6) a long term sedentary lifestyle, (7) menopause, (8) frequent stress, severe and acute stress, and/or chronic stress, (9) high blood levels of homocysteine (e.g., levels of about 25 μmol/L or more, such as about 30 μmol/L or more, such as about 40 μmol/L or more, such as about 35-100 μL or more (e.g., more than about 100 μmol/L)), (8) elevated levels of C-Reactive Protein (CRP) (e.g., about 3.2 mg/L or more, about 3.5 mg/L or more, about 3.6 mg/L or more, about 3.75 mg/L or more, about 3.9 mg/L or more, or about 4 mg/L or more), and/or (9) age (e.g., being about 40 or older, 45 or older, 50 or older, 60 or older, 65 or older, 70 or older, 75 or older, etc.). In one aspect, the invention provides a method of preventing or treating diabetes, pre-diabetes, or a related condition (or otherwise inducing IRBP-IR-associated signaling or binding to the IR), without upregulating one or more aspects of the IRACS pathway in a host having two or more of the above-recited exemplary HHDRFs, three or more of these HHDRFs, four or more of these HHDRFs, etc. (with or without the presence of a HCC). Other factors that also or alternatively may constitute HHDRFs include the presence of proteinuria (e.g., microalbuminuria); a ratio of plasma total to HDL cholesterol of 6 or higher; high triglyceride levels (e.g., triglyceride levels of above about 200 mg/dl, such as above about 250, 300, 350, or 400 mg/dl); hypothyroidism; high levels and/or particular allelic variants of plasminogen activator inhibitor-1 (PAI1); and/or abnormally high levels of fibrinogen.

### B. IRBPS

In the context of this invention, the term insulin receptor binding protein (IRBP) refers to a peptide or peptide-comprising molecule/composition (e.g., a peptide derivative) that (a) comprises at least one insulin receptor (IR)-binding amino acid sequence (IRBAAS) that (i) imparts or enhances insulin receptor agonist or partial agonist activity and (ii) is explicitly disclosed in or is encompassed by a formula disclosed herein and/or in one or more of the following patent documents: US Patent Application Publication Nos. 20030236190 and 20030195147; U.S. Patent Application No. US 09/538,038; U.S. Provisional Patent Applications 60/603,513 and 60/612,476; and International Patent Applications WO 01/72771, WO 03/027246, and WO 03/070747. These patent documents are collectively referred to herein as the “Prior Patent Documents” (“PPDs”) and are each explicitly incorporated by reference in their entirety herein.

### 1. IRBP Composition

Within the criteria described above, IRBPs can have any suitable composition. For example, IRBPs can comprise, and often advantageously comprise, non-essential, non-naturally occurring (or otherwise unusual), and/or non-L amino acid residues. Non-limiting examples of unusual amino acid residues that can be comprised in a derivative include, for example, 2-aminoadipic acid; 3-Aminoadipic acid; β-Alanine; β-aminopropionic acid; 2-Aminobutyric acid; 4-Aminobutyric acid; 6-Aminocaproic acid; 2-Aminoheptanoic acid; 2-Aminoisobutyric acid; 3-Aminoisobutyric acid, 2-Aminopimelic acid; 2,4-Diaminobutyric acid; Desmosine; 2,2′-Diaminopimelic acid; 2,3-Diaminopropionic acid; N-Ethylglycine; N-Ethylasparagine; Hydroxylysine; allo-Hydroxylysine; 3-Hydroxyproline; 4-Hydroxyproline; Isodesmosine; allo-Isoleucine; N-Methylglycine; N-Methylisoleucine; 6-N-Methyllysine; N-Methylvaline; Norvaline; Norleucine; and Ornithine. Additionally advantageous unusual amino acids relevant to particular aspects of the invention are described further elsewhere herein. Although included in the broad meaning of terms such as peptide and protein, it will be recognized that proteins comprising such unusual amino acid residues can be considered unique aspects of the invention as compared to peptides comprising combinations of the 20 typical and naturally occurring D amino acid residues.

IRBPs typically can be described as single-chain peptides or proteins. Generally speaking, terms such as “protein,” “polypeptide,” and “peptide” herein should be understood as referring to any suitable amino acid-based oligomeric/polymeric molecule of any suitable size and composition (e.g., with respect to the number of associated chains comprised thereby and number of individual amino acid residues contained therein), as well as origin (e.g., whether the molecule is obtained by recombinant expression, isolation from natural sources, production by solid phase synthesis, a combination of such methods, etc.). While such terms should not be construed as interchangeable in meaning, for sake of convenience, the terms peptide, protein, and polypeptide should be construed as providing support for one another, unless otherwise stated or clearly contradicted by context. For example, an individual reference to a “protein” should be construed as also providing equivalent literal support for an essentially identical aspect of the invention involving a “peptide” (a single chain protein of from 3 to about 50 amino acid residues) or “polypeptide” (a single chain protein of >about 50 amino acid residues in length), provided that such an understanding is reasonable and not clearly contradicted. However, this instruction does not imply that such polymeric molecules are not different from one another in certain aspects (e.g., in terms of formulation for oral delivery), such that in some instances a “peptide” provided by the invention (explicitly or by use of a term such as protein) may significantly differ from a “polypeptide.” Typically, a peptide or protein described in the context of this invention refers to an individual, primarily peptide bond-linked, amino acid polymer containing molecule (e.g., a single amino acid chain or a derivative thereof).

IRBPs and other proteins described here also may be derivatized proteins, which are further described elsewhere herein, unless otherwise stated or clearly contradicted by context. Protein derivatives and proteins can be associated with significantly different features, however, and also can be considered unique aspects of the invention. In other words, the “inclusion” of derivatives in the broadest meaning of the term “protein” is done for purposes of convenience in describing the various features of this invention, rather than to imply any sort of equivalence between such molecules.

IRBPs can be prepared by any suitable method. For example, IRBPs, particularly non-derivative IRBPs, can be produced as fusion proteins in any suitable expression system.

Methods and principles relevant to the production of recombinant fusion proteins are well known in the art and need not be discussed in detail here. Standard peptide synthesis can be used to generate IRBPs as well. Such recombinantly produced or synthesized peptides can further be subjected to derivation, conjugation, multimerization, etc. to form more complicated molecules within the scope of this invention. Multivalent IRBPs and IRBP fusion proteins also can be generated by conventional chemical linkage of amino acid chains and/or other moieties/substituent molecules. Again, such methods and the principles related thereto are well characterized in the art. IRBPs likewise can be purified by any suitable technique. For example, IRBP fusion proteins comprising particular purification “tags” (purification facilitating sequences or moieties) can be generated by known methods and used to obtain such molecules. For direct purification, methods such as differential electrophoresis, chromatography, centrifugation also can be used as can affinity (e.g., antibody-based) methods directed to the characteristics of a non-fusion protein IRBP. A number of such techniques are specifically described with respect to the production and purification of IRBPs in the PPDs.

The structure of IRBAASs, which typically are the primary distinguishing feature of IRBPs, are described in the following section.

### 2. IRBAAS Composition

As mentioned above, IRBPs are characterized by, among other things, inclusion of one or more IRBAASs, which can be, in turn, characterized by having a structure according to one of several structural formulas described here and/or in the PPDs. To better illustrate the invention, exemplary IRBAAS formulas and specific IRBAASs are described here. IRBPs can include any one or combination of IRBMS formulas provided here or in the PPDs. A number of specific types of combinations are described further herein.

a. Formula 1 Type IRBAASs

In one exemplary aspect, one or more of the inventive methods of this invention can be practiced with an IRBP that comprises one or more IR-binding portions that consist or consist essentially of an amino acid sequence according to the formula X₁ X₂ X₃ X₄ X₅ wherein X_{1′}X_{2′}, X₄ and X₅ are aromatic amino acids, and X₃ is any polar amino acid (Formula 1) or a sequence according to a similar formula (Formula 1-like, or FOL, sequences) described here or in the PPDs (FOLs may differ from Formula 1 in that, for example, X₃ represents any suitable amino acid residue, which may be any of the 20 common naturally occurring amino acid residues; an unusual amino acid residue that is resistant to enzymatic degradation; or even a non-amino acid residue moiety). Suitability in terms of amino acid residues or other moieties that substitute for amino acid residues (or lack of residues at particular positions in a formula) for highly variable positions in IRBMS formulas provided herein means a residue, moiety, etc. that allows the IRBP to bind an IR and detectably induce, promote, or enhance IR agonist activity in a cell (e.g., in a chordate cell, typically a mammalian cell, more typically a human cell, in vitro, ex vivo, or in vivo) and desirably at therapeutic levels in a mammalian (e.g., human) subject. Those that work routinely in the field of peptide production will readily be able to determine suitable choices given known considerations for peptide structure (many of which are described elsewhere herein and in the PPDs), the guidance given herein and in the PPDs in terms of exemplary sequences, and through use of no more than routine experimentation.

In a more particular aspect, inventive methods of the invention may be practiced with a Formula 1 sequence, wherein X₁ and X₅ are phenylalanine and X₂ is tyrosine (Formula 1.1). In one aspect, methods can be practiced with an IRBP comprising a Formula 1 IRBAAS wherein X₃ is a small polar amino acid. In a more particular exemplary aspect, X₃ is aspartic acid, glutamic acid, glycine, or serine (in a yet further particular aspect, X₃ is aspartic acid or glutamic acid). In another facet, methods can be practiced with an IRBP comprising a Formula 1 IRBMS or FOL wherein X₄ also or alternatively is a tryptophan residue. Thus, in one aspect, methods can be practiced with an IRBP that comprises at least one sequence according to the formula FYX₃WF (SEQ ID NO:1), wherein X₃ can be any suitable residue (including an unusual residue) or an organic moiety. Exemplary Formula 1 IRBAASs according to this specific formula include FYDWF (SEQ ID NO:2), FYEWF (SEQ ID NO:3), and FYGWF (SEQ ID NO:4).

In any case, residues flanking the core Formula 1 or FOL motif, as described above, are typically selected and included to ensure/enhance IR agonist or partial agonist activity. In a typical aspect, the flanking residues can be characterized by a formula X₆ X₇ X₈ X₉ X₁₀ (or X₉₃ X₉₄ X₉₅ X₉₆ X₉₇ in the case of certain PPDs) wherein X₆ typically is an alanine, valine, aspartic acid, glutamic acid, and arginine; X₇ and X₁₀ represent any suitable amino acid residues; X₈ is glutamine, glutamic acid, alanine or lysine (and most typically glutamine or glutamic acid). X₉ is typically a hydrophobic or aliphatic amino acid and commonly selected from leucine, isoleucine, valine, or tryptophan (and very often is leucine). Hydrophobic residues, especially tryptophan at X₉, may be used to enhance IR selectivity.

In another particular exemplary aspect, inventive methods may be practiced with an IRBP that comprises one or more sequences that consist or consist essentially of a sequence according to the formula Xaa₁ Tyr Xaa₃ Trp Xaa₅, wherein (a) Xaa₁, Xaa₅, or both represent either (i) degradation-resistant unusual amino acid residues or degradation-resistant chemical moieties or (ii) Phe residues, and (b) Xaa₃ is a degradation-resistant unusual amino acid residue, a non-amino acid residue degradation resistant chemical moiety, or any suitable other amino acid residue (Formula 1A). In a more particular aspect, various methods of the invention can be practiced by use of an IRBP comprising an IRBMS that consists or consists essentially of a sequence according to the formula Xaa₁ Tyr Xaa₃ Trp Xaa₅ Xaa₆ Xaa₇ Xaa₈ Xaa₉, wherein Xaa₆ is any suitable amino acid residue (typically a residue other than Asp or Asn); Xaa₇ is any suitable residue; Xaa₈ is selected from Gln, Glu, Ala, and Lys; and Xaa₉ represents a hydrophobic amino acid (Formula 1B). In still a more particular aspect, inventive methods described here can be practiced with an IRBP comprising an IRBAAS that consists or consists essentially of a sequence according to the formula Xaa₁ Tyr Xaa₃ Trp Xaa₅ Glu Arg Gln Leu (SEQ ID NO:5), wherein Xaa₁, Xaa₃, and Xaa₅ are defined as in Formula 1A (Formula 1C). In yet an even more specific aspect, various inventive methods can be practiced using an IRBP comprising at least one IRBAAS that consists or consists essentially of a sequence according to the formula Xaa₁ Tyr Xaa₃ Trp Xaa₅ Glu Arg Gln Leu Gly (SEQ ID NO:6), wherein Xaa₁, Xaa₃, and Xaa₅ are defined as in Formula 1a (Formula 1D). In another particular facet, inventive methods can be practiced with an IRBP that comprises at least one IRBAAS that consists or consists essentially of a sequence according to the formula Xaa₁ Tyr Gly Trp Xaa₅ Glu Arg Gln Xaa₉ Gly (SEQ ID NO:7), wherein Xaa₁ is a Phe or degradation-resistant residue/moiety; Xaa₅ is a Phe or degradation-resistant moiety/residue; and Xaa₉ is any suitable residue (and typically a Leu) (Formula 1E). In a further aspect, inventive methods can be practiced with an IRBP that comprises at least one IRBAAS that consists or consists essentially of a sequence according to the formula Xaa₁ Tyr Xaa₃ Trp Xaa₅ Glu Arg Gln Leu Gly (SEQ ID NO:8), wherein Xaa₁ and Xaa₅ are defined as in Formula 1e, and Xaa₃ is a Gly or His residue (Formula 1F).

In still another exemplary aspect, inventive methods can be practiced with an IRBP that comprises at least one IRBAAS that consists or consists essentially of a sequence according to the formula Xaa₁ Tyr Xaa₃ Trp Xaa₅ Xaa₆ Xaa₇ Xaa₈ Xaa₉ Xaa₁₀, wherein Xaa₁ is a Phe or degradation-resistant residue/moiety; Xaa₅ is a Phe or degradation-resistant moiety/residue; Xaa₃ is any suitable residue; Xaa₆-Xaa₈ are any suitable residues; Xaa₉ is any suitable residue or is missing; and Xaa₁₀ is a hydrophobic residue (Formula 1G). In a particular aspect, inventive methods may be practiced using an IRBP that comprises IRBAASs that consist or consist essentially of a sequence according to Formula 1G, wherein one or more (or all) of Xaa₆₋₈ and also or alternatively Xaa₉ (is present) are hydrophilic residues (e.g., Glu, Gln, Asp, Lys, or Arg residues). In one such aspect, most, or all, of such residues are hydrophilic. In another particular variant, the invention provides various methods of using an IRBP that comprises IRBAASs consisting or consisting essentially of a Formula 1G sequence wherein the sequence also or alternatively is characterized by Xaa₃ representing a degradation-resistant residue or moiety. An example of such an IRBAAS is an IRBAAS according to the more particular formula Phe Tyr Xaa₃ Trp Phe Glu Arg Gln Leu (SEQ ID NO:9), wherein Xaa₃ represents an enzyme degradation-resistant amino acid residue or moiety. In still another variant of any of the foregoing IRBMS aspects, Xaa₃ represents a residue selected from Glu, Gly, or His. In particular aspects, Xaa₁₀ is a Leu, Val, Met, Ile, or Gly residue. In an even more particular aspect, Xaa₁₀ represents either a Leu or Gly residue. In one aspect, methods of using an IRBP comprising a sequence according to any of the foregoing formulas is provided wherein Xaa₉ and Xaa₁₀ both represent hydrophilic residues; such as, e.g., Leu and Gly, respectively.

b. Formula 2 Type IRBAASs

In another exemplary aspect, various inventive methods provided here can be practiced with an IRBP that comprises at least one IRBAAS that consists or consists essentially of a sequence according to the formula X₁₁ X₁₂ X₁₃ X₁₄ X₁₅ X₁₆ X₁₇ X₁₈ wherein X₁₁ and X₁₂ are aromatic amino acids, X₁₃, X₁₄, X₁₆ and X₁₇ are any suitable amino acid, and X₁₅ and X₁₈ are hydrophobic amino acids (Formula 2). In a particular aspect, X₁₁ and X₁₂ are phenylalanine or tyrosine (in a specific facet, X₁₁ is phenylalanine and X₁₂ is tyrosine) and/or X₁₅ and X₁₈ are hydrophobic amino acids (in a specific facet X₁₅ and X₁₈ are selected from isoleucine, phenylalanine, tryptophan or methionine—and in an even more particular aspect X₁₈ is selected from leucine or isoleucine and X₁₅ is isoleucine). Inventive methods also may be practiced with IRBPs comprising one or more Formula 2-like (FTL) IRBAASs, which differ from Formula 2 by, for example, inclusion of one or more unusual enzyme degradation resistant amino acid residues and/or organic moieties (e.g., at positions X₁₃, X₁₄, X₁₆ and/or X₁₇).

Another Formula 2 type IRBAAS is X₁₁₅ X₁₁₆ X₁₁₇ X₁₁₈ F Y X₈ Y F X₁₁ X₁₂ L X₁₁₉ X₁₂₀ X₁₂₁ X₁₂₂ (SEQ ID NO:10), wherein X₁₁₅-X₁₁₈ and X₁₁₈-X₁₂₂ may be any amino acid which allows for binding to IR. X₁₁₅ is typically selected from the group consisting of tryptophan, glycine, aspartic acid, glutamic acid, and arginine; and commonly are selected from aspartic acid, glutamic acid, glycine, and arginine (tryptophan being most common). X₁₁₆ commonly is an amino acid selected from the group consisting of aspartic acid, histidine, glycine, and asparagine. X₁₁₇ and X₁₁₈ are typically glycine, aspartic acid, glutamic acid, asparagine, or alanine. More commonly, X₁₁₇ is glycine, aspartic acid, glutamic acid and asparagine whereas X₁₁₈ is more commonly glycine, aspartic acid, glutamic acid or alanine. X₈ when present in the Formula 2A motif is typically arginine, glycine, glutamic acid, or serine. X₁₁ when present in the Formula 2A motif is usually glutamic acid, asparagine, glutamine, or tryptophan, but most commonly glutamic acid. X₁₂ when present in the Formula 2A motif usually is aspartic acid, glutamic acid, glycine, lysine or glutamine, but most commonly is aspartic acid. X₁₁₉ is usually glutamic acid, glycine, glutamine, aspartic acid or alanine, but most commonly is glutamic acid. X₁₂₀ is typically glutamic acid, aspartic acid, glycine or glutamine, but most commonly is glutamic acid. X₁₂₁ is usually tryptophan, tyrosine, glutamic acid, phenylalanine, histidine, or aspartic acid, and most typically tryptophan or tyrosine. X₁₂₂ is often glutamic acid, aspartic acid or glycine; and regularly is glutamic acid.

Amino terminal and carboxy terminal extensions associated with type Formula 2 sequences may be represented as X₉₈ X₉₉ Formula 2 X₁₀₀, wherein X₉₈ is optionally aspartic acid and X₉₉ is independently an amino acid selected from the group consisting of glycine, glutamine, and proline. The presence of an aspartic acid at X₉₈ and a proline at X₉₉ is associated with an enhancement of IR binding. A hydrophobic amino acid typically is present at X₁₀₀ and an aliphatic amino acid is ore typical (leucine being often present at this position). Negatively charged amino acids are regularly at both the amino and carboxy terminals of Formula 2A.

In another aspect, the inventive methods are practiced with an IRBP that comprises a Formula 2 type IRBAAS that consists or consists essentially of the sequence Ser Glu Gly Phe Tyr Asn Ala Ile Glu Leu Leu Ser (SEQ ID NO:11) (Formula 2B).

c. Formula 6 Type IRBAASs

In another aspect, inventive methods can be practiced with IRBPs that comprise at least one IRBMS that consists or consists essentially of a sequence according to the formula X₆₂ X₆₃ X₆₄ X₆₅ X₆₆ X₆₇ X₆₈ X₆₉ X₇₀ X₇₁ X₇₂ X₇₃ X₇₄ X₇₅ X₇₆ X₇₇ X₇₈ X₇₉ X₈₀ X₈₁, wherein X₆₂, X₆₅, X₆₈, X₆₉, X₇₁, X₇₃, X₇₆, X₇₇, X₇₈, X₈₀, and X₈₁ may be any amino acid; X₆₃, X₇₀, X₇₄ are hydrophobic amino acids; X₆₄ is a polar amino acid; X₆₇ and X₇₅ are aromatic amino acids; and X₇₂ and X₇₉ are preferably cysteines capable of forming a loop (Formula 6). In other aspects, inventive methods are practiced with a similar (Formula 6-like sequence or FSL sequence), but which differs in one or more respects (e.g., the lack of one or more cysteines in the sequence and accordingly of any loop structure). In particular aspects, inventive methods can be practiced with IRBPs comprising a Formula 6 sequence wherein X₆₆ is a residue other than glutamine or valine and commonly is glutamic acid; X₆₃, X₇₀, and X₇₄ are hydrophobic amino acids; X₆₃ is leucine, isoleucine, methionine, or valine (and most commonly leucine); X₇₀ and X₇₄ are typically valine, isoleucine, leucine, or methionine (X₇₄ is most commonly valine); X₆₄ is a polar amino acid (commonly aspartic acid or glutamic acid and most commonly glutamic acid); X₆₇ and X₇₅ are aromatic amino acids (tryptophan is common at X₆₇ and X₇₅ is commonly tyrosine or tryptophan and most commonly tyrosine); and X₇₂ and X₇₉ are cysteines (loop forming cysteines can be shifted in position in a similar sequence, where desired). An example of a more particular Formula 6 type formula is X₆₂ L X₆₄ X₆₅ X₆₆ W X₆₈ X₆₉ X₇₀ X₇₁ C X₇₃ X₇₄ X₇₅ X₇₆ X₇₇ X₇₈ C X₈₀ X₈₁ (SEQ ID NO:12).

In another aspect, inventive methods can be performed with IRBPs that comprise an IRBAAS that consists or consists essentially of a sequence according to the formula Xaa₁ Leu Glu Xaa₄ Glu Trp Xaa₇ Xaa₈ Xaa₉ Xaa₁₀ Xaa₁₁ Xaa₁₂ Val Tyr Xaa₁₅ Xaa₁₆ Xaa₁₇ Xaa₁₈ (SEQ ID NO:13), wherein Xaa₁, Xaa₄, Xaa₇, Xaa₈, Xaa₉, Xaa₁₀, Xaa₁₂, Xaa₁₅, Xaa₁₆, and Xaa₁₇ are any suitable amino acid residues and Xaa₁₁, Xaa₁₈, or both are any suitable residue other than Cys (Formula 6A). In a more particular aspect, inventive methods can be practiced with IRBPs that comprise an IRBAAS that consists or consists essentially of a sequence according to formula 6a, wherein Xaa₁₁ is an Ala or Glu, Xaa₁₈ is an Ala or Glu, or both Xaa₁₁ and Xaa₁₈ are, independently, Ala or Glu residues (Formula 6B). In a particular aspect, Xaa₁₁ and/or Xaa₁₈ are Ala residues. In a further particular aspect, methods can be practiced using an IRBP that comprises an IRBAAS that consists essentially or consists of a sequence according to the formula Ser Leu Glu Glu Glu Trp Ala Gln Ile Glu Xaa₁₁ Glu Val Trp Gly Arg Gly Xaa₁₈ (SEQ ID NO:14), wherein Xaa₁₁ and/or Xaa₁₈ represent any suitable residue other than Cys (Formula 6C). In a more particular aspect, inventive methods can be practiced with an IRBP that comprises at least one IRBAAS that consists or consists essentially of a sequence according to Formula 6C, wherein Xaa₁₁ and/or Xaa₁₈ represent Ala residues (Formula 6D).

In a further aspect, various inventive methods can be practiced using IRBPs that comprise an IRBMS that consists or consists essentially of a sequence according to one or more of Formulas 6A-6D wherein the C-terminus of the sequence is joined to a C-terminal sequence according to the formula Xaa₁₉ Xaa₂₀ Xaa₂₁, wherein Xaa₂₁ is not a hydrophobic or aliphatic residue and Xaa₁₉ and Xaa₂₀ are any suitable residues. In a more particular aspect, Xaa₂₁ is a Glu residue. In yet another particular aspect, the C-terminal sequence is also or alternatively characterized by Xaa₁₉ representing a Pro residue, Xaa₂₀ representing a Ser residue, or both. Examples of Formula 6D IRBMS-containing IRBPs include peptides S574 (SLEEEWAQIEAEVWGRGAPSESFYDWFERQLG—SEQ ID NO:15) and S727 (Ac-SLEEEWAQIEAEVWGRGAPSESFYDWFERQLG-NH2—SEQ ID NO:16).

In a further aspect, various inventive methods can be practiced using an IRBP that comprises at least one IRBAAS that consists or consists essentially of a sequence according to one or more of Formulas 6A-6D, typically with the inclusion of a C-terminal sequence as described in the preceding paragraph, wherein the N-terminal residue of the sequence (Xaa₁) is acylated and, more typically, acetylated. Typically, Xaa₁ represents an acetylated Ser residue. Peptide S727 is an example of an IRBP comprising such an IRBAAS.

In yet another aspect, various methods provided here can be practiced with an IRBP that comprises at least one IRBAAS that consists or consists essentially of a sequence according to the formula Xaa₁ Leu Glu Xaa₄ Glu Trp Xaa₇ Xaa₇ Xaa₉ Xaa₁₀ Xaa₁₁ Xaa₁₂ Val Tyr Xaa₁₅ Xaa₁₆ Xaa₁₇ Xaa₁₈ (SEQ ID NO:17), wherein (a) Xaa₁₁ and/or Xaa₁₈ are Cys residues or other suitable amino acid residues and (b) one or more of Xaa₄, Xaa₇, Xaa₈, Xaa₁₅, and Xaa₁₇ represent degradation-resistant unusual amino acid residues and/or moieties (Formula 6E). In one aspect, such an IRBAAS comprises at least two degradation-resistant unusual residues or moieties.

In an additional exemplary aspect, methods provided by this invention can be practiced with IRBPs that comprise at least one IRBAAS that consists or consists essentially of a sequence according to the formula Ser Leu Glu Glu Glu Trp Ala Gln Ile Xaa₁₀ Xaa₁₁ Glu Val Trp Gly Arg Gly Xaa₁₈ (SEQ ID NO:18), wherein Xaa₁₀ is Glu or Gln and Xaa₁₁ and Xaa₁₈ are any suitable residues (Formula 6F). In one aspect, the invention provides IRBPS that comprise an IRBMS wherein Xaa₁₁ and/or Xaa₁₈ are Cys residues. In a more particular aspect, both Xaa₁₁ and/or Xaa₁₈ are Cys residues. In an alternative aspect, both Xaa₁₁ and Xaa₁₈ are characterized as any suitable residue other than Cys residues. In a particular facet of such an aspect, Xaa₁₁ and/or Xaa₁₈ can be, for example, independently Ala or Glu residues.

In still another exemplary aspect, various inventive methods provided here may be practiced using an IRBP that comprises at least one IRBAAS that consists or consists essentially of a sequence according to the formula Xaa₁ Leu Glu Xaa₄ Glu Trp Xaa₇ Xaa₈ Xaa₉ Xaa₁₀ Xaa₁₁ Xaa₁₂ Val Tyr Xaa₁₅ Xaa₁₆ Xaa₁₇ Xaa₁₈ (SEQ ID NO:19), wherein Xaa₁, Xaa₄, Xaa₇, Xaa₈, Xaa₉, Xaa₁₀, Xaa₁₂, Xaa₁₅, Xaa₁₆, and Xaa₁₇ are any suitable amino acid residues; Xaa₁₈ is Cys or a suitable residue other than Cys (e.g., Ala or Glu); and Xaa₁₁ is Cys or a suitable residue other than Cys (e.g., Ala or Glu) (Formula 6G). In one version, Xaa₁₈ is Cys. In one facet, inventive methods can be practiced with an IRBP comprising a Formula 6G IRBMS wherein Xaa₁₀ also or alternatively is Glu or Gln.

In yet a further exemplary facet, inventive methods can be practiced using an IRBP that comprises at least one IRBAAS that consists or consists essentially of a sequence according to one or more of Formulas 6A-6G, wherein the sequence is characterized as not forming internal Cys-Cys bonds, not comprising a Cys residue, and/or not forming a cyclic peptide conformation under typical physiological conditions.

The methods of this invention are not limited to the above-described Formula 1 type IRBMSs, Formula 2 type IRBMSs, and Formula 6 IRBAASs, but may be practiced with IRBPs comprising any suitable IRBAASs described in the PPDs (e.g., in one aspect the inventive methods can be practiced with an IRBP comprising a Formula 4 IRBAAS as described in the PPDs).

In certain aspects, methods described herein can be advantageously practiced with IRBPs that comprise at least two IRBAASs, which can include (a) two or more identical types of IRBAASs (e.g., two Formula 1 type IRBAASs) which, in turn can be identical or not identical, (b) two different types of IRBAASs, or (c) both. Commonly, methods of the invention are practiced with IRBPs that comprise at least two different types of IRBAASs, such that the IRBP is specific for different sites on an IR and also can be considered multivalent. Multivalent IRBPs, and multivalent and multispecific IRBPs, and uses thereof in the context of this invention, are further described in the following section.

### 3. Multivalent and Multispecific IRBPs

As described above, in one aspect, methods of this invention may be practiced with IRBPs that comprise two or more IRBAASs, which, respectively, bind to one or more sites of IR (e.g., Site 1 or Site 2). Such multivalent IRBPs can be produced by standard fusion protein expression technology, chemical conjugation, or any other suitable technique for producing a multivalent IRBP (various methods are described in detail in the PPDs). In one aspect, various inventive methods are practiced using a multivalent IRBP that comprises at least one Site 1-binding amino acid sequence and at least one site-2 binding amino acid sequence. Such IRBPs can be described as multispecific as well as multivalent. In another aspect, inventive methods are practiced using a multivalent IRBP comprising two or more sequences that specifically bind to the same site on IR.

The specificities of various IRBAASs are set forth in the PPDs and/or are readily determinable with routine experimentation. In general, Formula 1 type IRBAASs are specific for IR Site 1, whereas Formula 6 Type IRBAASs and Formula 2 Type IRBMSs bind to IR Site 2.

In general, multispecific IRBPs can be characterized on the basis of little or no competition between the Site 1 and Site 2 binding IRBMSs comprised therein.

IRBPs comprising two or more IRBMSs that bind to the same site to form a multivalent ligand may be useful to produce molecules that are capable of cross-linking together multiple receptor units. Multivalent ligands may also be constructed to combine amino acid sequences which bind to different sites.

Additional aspects of particular multivalent IRBMSs are separately discussed in the following subsections.

a. Orientation of IRBAASs

In one aspect, various methods provided by the invention can be practiced with IRBPs that comprise two or more IRBAASs that are covalently linked at their N-termini or C-termini to form N-N, C-C, N-C, or C-N linked regions or peptides. These may be directed to the same IR site-Site 1-Site 1 or Site 2-Site 2 combinations. Alternatively, Site 1-Site 2 or Site 2-Site 1 combinations are provided. Site 2-Site 1 combinations are typically IR agonists. Any IRBP comprising such a combination of IRBMSs can be referred to as a “dimer.”

In specific aspects, Site 1-Site 2 and Site 2-Site 1 orientations are possible. In addition, N-terminal to N-terminal (N-N); C-terminal to C-terminal (C-C); N-terminal to C-terminal (N-C); and C-terminal to N-terminal (C-N) linkages are possible. Accordingly, IRBPs may be oriented Site 1 to Site 2, or Site 2 to Site 1, and may be linked N-terminus to N-terminus, C-terminus to C-terminus, N-terminus to C-terminus, or C-terminus to N-terminus. In certain cases, a specific orientation may be preferable to others, for example, for maximal agonist or antagonist activity.

The orientation and linkage of the “monomer” subunits (portions consisting or consisting essentially of various IRBAASs) has been found to dramatically alter IRBP dimer activity. In particular, certain Site 1/Site 2 heterodimer sequences show agonist or antagonist activity at IR, depending on orientation and linkage of the constituent monomer “subunits” (IRBAASs or sequences comprising or consisting essentially thereof). For example, a Site 1-Site 2 orientation (C-N linkage) shows antagonist activity at IR and accordingly is not typically useful in the context of this invention. In contrast, a Site 2-Site 1 orientation (C-N linkage) shows potent agonist activity at IR. Similarly, Site 1-Site 2 (C-N linkage) heterodimers show antagonist activity at IR (and accordingly are typically not suitable for the inventive methods provided here), while Site 1-Site 2 (C-C or N-N linkage) heterodimers show agonist activity. Further details concerning the association of orientation, linkage, and activity of multivalent IRBPs are provided in the PPDs or can readily be determined with routine experimentation (in general it should be noted that not all of the PPDs use the same terminology as used herein with respect to describing IRBPs).

Whether produced by recombinant gene expression or by conventional chemical linkage technology, various IRBAASs may be coupled through linkers of various lengths and IRBPs comprising such linkers may be advantageously used in aspects of the invention provided here. In one aspect, IRBPs for use in methods described here can be characterized by the inclusion of no linker or at most a very short linker between IRBAASs (e.g., a linker consisting of less than about 5 residues, such as 0, 1, or 2 residues). An intra-IRBAA “linker” typically consists of one or a few small and/or flexible typical amino acid residues, such as a Gly, a Val, and/or a Ser residue; one or more digestive enzymatic degradation-resistant unusual amino acid residues; one or more degradation-resistant non-amino acid moieties; or a combination of any thereof.

Methods of the invention may also be practiced using IRBPs that comprise one or more IRBMSs linked to additional non-IRBMS sequences (e.g., sequences that promote stabilization, targeting (such as a cholera toxin B fusion partner), detection (e.g., a green fluorescent protein (GFP) sequence, firefly luciferase sequence, epitope tag sequence, an enzyme substrate or active enzyme sequence; or similar sequence), stabilization (e.g., a ubiquitin sequence for improved production in E. coli or other stabilizing sequence), and/or purification (e.g., a hexa-histidine sequence or other His-tag; an epitope tag; a glutathione S-transferase (GST) sequence; or the like) of the IRBP or that impart additional pharmacological/biological functionality (such as binding to a second target other than IR) in the context of a fusion protein. A linker between IRBAAS(s) and the non-IRBAAS sequence(s) may be significantly longer than those commonly used to link IRBAASs, particularly in the case of a fusion protein that comprises one or more secondary ligand-binding sequences/domains. Principles and techniques relevant to the selection and inclusion of such linker sequences are well known in the art. A specific example of such an IRBP fusion protein is embodied in IRBP S860, which comprises a His-tag and an ubiquitin fusion partner portion.

b. N-terminal Acetylated/C-Terminal Amidated Multivalent IRBPs

Various methods of the invention can be advantageously practiced using IRBPs that are characterized by N-terminal acylation, typically acetylation, of an included IRBAAS and/or C-terminal amidation of an included IRBAAS. For example, the invention in one aspect provides an IRBP that comprises one N-terminal and acetylated IRBAAS and a different C-terminal and amidated IRBAAS. Such modifications can surprisingly improve the “modified” molecule in terms of stability and/or IR binding (as compared to an essentially identical molecule lacking the modification(s)). An IRBP in this context can comprise one or more IRBMS as described herein (e.g., an IRBAA according to Formula 1-Formula 1G) or a sequence (Formula) of one or more of the insulin-binding peptides described in the PPDs (e.g., a Formula 4 sequence as described in US 20030236190). Typically, the N-terminal acetyl and/or C-terminal amide are directly linked to the termini of IRBMSs. However, in the case of addition variants/fusion proteins, these substituents can be associated with non-IRBAAS residues that are in turn directly or indirectly linked to “internally positioned” (or “internal”) IRBAASs.

In a particular aspect, methods of the invention can be practiced with IRBPs comprising a sequence that consists or consists essentially of (I) either Formula 6 (or a particular aspect thereof) or one or more of Formulas 6A-6G and (II) (a) an IRBAAS according to Formula 1 (or particular aspect thereof) or one or more of Formulas 1A-1G or (b) an IRBAAS according to Formula 2 (or particular aspect thereof) or Formula 2A, wherein the IRBPs can be characterized by N-terminal acetylation and/or C-terminal amidation. Typically, such IRBPs are “dimers” of two of such Formula 6/Formula 6-like (i.e., a sequence according to Formula 6 or Formulas 6A-6G) and non-Formula-6-like sequences (i.e., Formula 2, Formula 2a, or Formula 1a-1 g sequence). Typically, such IRBPs are directly linked or separated by a very short linker (e.g., a linker of 1-3 residues or moieties). Typically, such dimers are oriented Site 2-Site 1 (C-N linkage).

Methods of this invention also can include the use of IRBPs provided in the PPDs but that are modified by such N-terminal acetylation and/or C-terminal amidation modifications (e.g., a Formula 1-Formula 4 dimer that comprises one or both types of such modifications) represents another feature of the invention.

c. Degradation-Resistant Multivalent Derivatives

As mentioned above, various methods provided here can be advantageously practiced using IRBPs, typically multivalent IRBPs, that include digestive enzyme degradation-resistant amino acid residues and/or moieties.

In one exemplary aspect, various methods provided here may be practiced with degradation-resistant IRBPs that comprise one or more IRBAASs according to one or more of Formulas 6A-6G and at least one Formula 2 sequence (see, e.g., US 20030236190) or Formula 2A sequence, arranged in a Site 2-Site 1 orientation (C-N linkage). In a particular aspect, methods may be practiced using IRBPs that comprise a single IRBMS according to one or more of Formulas 6A-6G or Formula 6 and a single Formula 2 or Formula 2A sequence comprising one or more degradation-resistant unusual amino acid (M) residues and/or non-AA moieties located between the IRBAASs, at one or both termini of the IRBP, or both, wherein the sequence optionally is further characterized by inclusion of one or two linker residues between the respective IRBAASs, which may be in place of or in addition to one or more degradation-resistant moiety or residue linkers.

In another facet, inventive methods provided here may be practiced using one or more degradation-resistant IRBPs that comprise a Formula 6 IRBAAS and a Formula 2 IRBAAS (or any particular IRBMS described in association with these formulas here or in the PPDs), wherein the IRBP comprises a degradation resistant unusual residue or moiety located between the IRBAASs or at either or both termini of the dimer. Such IRBPs typically have a Site 2-Site 1 orientation (C-N linkage).

In another aspect, inventive methods provided here can be practiced using one or more IRBPs that comprise at least one IRBAAS according to one or more of Formulas 1A-1G and at least one IRBMS according to one or more of Formulas 6A-6G. In another aspect, the invention provides IRBPs that comprise at least one IRBMS according to Formula 1 (or any particular example or aspect thereof as provided here or in the PPDs) and at least one IRBMS of Formulas 6A-6G. In still a further aspect, inventive methods can be practiced using one or more IRBPs that comprise at least one Formula 6 sequence (or particular example or aspect thereof as described here or in the PPDs) and at least one sequence according to one or more of Formulas 1A-1G. In yet an additional aspect, inventive methods provided here can be practiced using IRBPs according to any of the foregoing aspects of this paragraph, wherein the IRBP comprises one or more degradation-resistant unusual amino acid residues and/or degradation-resistant moieties located between the Formula 1 type IRBMS and the Formula 6 type IRBMS, at one or both termini of the IRBP, or a combination thereof. Methods can be, e.g., performed using a “dimer” IRBP exhibiting one or more of the features described in this paragraph. Such IRBPs typically exhibit a Site 2-Site 1 orientation (C-N linkage).

In still another aspect, inventive methods provided here an be practiced using an IRBP comprising a Formula 1 IRBAAS and a Formula 6 IRBAAS, which typically is a “dimer” thereof (and typically Site 2-Site 1 oriented (C-N linkage)) according to the prior patent documents (see, e.g., US 20030236190), wherein the IRBP comprises at least one degradation-resistant unusual amino acid residue and/or moiety between the IRBAASs and/or at one or both termini of the IRBP.

In a particular exemplary aspect, inventive methods are practiced using one or more IRBPs that comprise a Formula 6 type IRBMS (i.e., a Formula 6 sequence or FSL) and a Formula 1 IRBMS or FOL IRBAAS, oriented and linked as described above, wherein one or more degradation resistant moieties or residues are present at positions 5, 7, and/or 8 of the Formula 6 or FSL sequence, one or two degradation-resistant moieties or residues are present at positions 1 or 2 of the Formula 1 or FOL sequence, or both. Such IRBPs also can further comprise N-terminal and/or C-terminal blocking groups (e.g., acetyl and amide groups, respectively).

Any of the IRBPs described with respect to the methods provided herein can comprise terminally and/or internally positioned acyl derivatives linked to the amino acid sequence backbone thereof (e.g., to a Formula 2, Formula 6, and/or Formula 1 sequence and/or to one or more non-IRBAAS sequences) that also may increase the stability of the peptides. An acyl derivative in this context can be, for example, a C₁₂-C₂₂ carboxylic or dicarboxylic acid substituent (each sub-range and member hereof representing an individual aspect)). Such IRBPs can exhibit, for example, enhanced albumin binding/association, which in turn imparts increased in vivo half-life, as compared to other IRBPs and/or insulins. Other forms of acylation of IRBPs also can be suitable (e.g., as mentioned elsewhere herein).

In a particular aspect, inventive methods described here may be practiced with one or more IRBPs comprising a Formula 1 or FOL IRBAAS and a Formula 6 or FSL IRBMS (typically characterized by Site 2-Site 1 orientation, C-N linkage) wherein the Xaa₇ position of the Formula 6/FSL sequence is substituted with (represented by) a degradation-resistant residue or moiety (e.g., an Aib) and the Xaa₁ residue of the Formula 1 or Formula 1-like sequence is substituted with a degradation-resistant residue or moiety (e.g., a Dip). An example of such an IRBP is embodied in IRBP S873.

In another facet, various inventive methods described here can be practiced with IRBPs comprising a Formula 1 type IRBAAS and a Formula 6 type IRBAAS (typically characterized by Site 2-Site 1 orientation, C-N linkage), wherein the Xaa₅, Xaa₇, and/or Xaa₈ position of the Formula 6 type sequence is/are substituted with (i.e., represent) a degradation-resistant residue or moiety and the Xaa₁ and/or Xaa₅ residue(s) of the Formula 1 type sequence is/are substituted with (i.e., represent) a degradation-resistant residue or moiety.

With respect to any of the foregoing, the reference to a degradation-resistant residue or moiety at the termini of the IRBP should be understood as typically referring to the region defined by at least two IRBMSs; although in cases of variants that are modified by additions at one or both termini, such degradation-resistant residues/moieties may be associated with residues “outside” the context of the external IRBAASs themselves.

An unusual degradation-resistant amino acid residue or degradation-resistant moiety can be any suitable type of such a residue or moiety. Examples of unusual degradation-resistant residues include sarcosine, diphenylalanine, aminoisobutyric acid, D-arginine, and N-methylphenylalanine. Additional examples of such residues and degradation resistant moieties suitable for inclusion in multivalent IRBPs are described elsewhere herein and in the PPDs.

In another aspect, various inventive methods provided here can be practiced using one or ore IRBPs comprising at least two different IRBMSs according to different formulas (as provided herein and/or in the prior patent documents), which can be characterized by inclusion of at least two degradation-resistant amino acid residues or moieties positioned and selected such that that each such IRBP more degradation-resistant than a similar sequence lacking the residues/moieties.

In a further aspect, inventive methods can be practiced using one or more IRBPs according to any of the formulas provided here or the PPDs, wherein the IRBP also can be characterized by the presence of N-terminal acetylation and/or C-terminal amidation (e.g., of the terminal residues of IRBAASs contained therein).

### 4. Exemplary IRBAASs and IRBPs

A number of suitable, specific, and illustrative IRBAASs that can be included in IRBPs for practicing inventive methods described herein are provided in the PPDs. To better illustrate the invention, a nonlimiting list of exemplary IRBMSs also is provided in Table 1:

TABLE 1
Exemplary IRBPs

| | | SEQ |
|---|---|---|
| Refer- | | ID |
| ence | Sequence | NO |
| S519 | SLEEEWAQVECEVYGRGCPSGSLDESFYDWFERQLG | 20 |
| S557 | SLEEEWAQIECEVYGRGCPSESFYDWFERQL | 21 |
| S582 | SLEEEWAQIECEVWGRGCPKGFYGWFRRRG | 22 |
| S597 | Ac-SLEEEWAQIECEVYGRGCPSESFYDWFERQL | 23 |
| S634 | Ac-SLEEEWAQIQCEVWGRGCPSESFYDWFEAQLHA | 24 |
| S636 | Ac-SLEEEWAQIQCEVWGRGCPSESFYDWFERQL | 25 |
| S642 | Ac-SLEEEWAQIQCEVWGRGCQRPEPFYDWFERQL | 26 |
| S665 | Ac-SLEEEWAQIECEVYGRGCPSESFYHWFERQL | 27 |
| S671 | Ac-SLEEEWAQIQCEVWGRGCPSESFYDWFERQLG | 28 |
| S726 | Ac-SLEEEWAQIECEVWGRGCPSEGFYNAIELLS | 29 |
| S727 | Ac-SLEEEWAQIEAEVWGRGAPSESFYDWFERQLG-NH2 | 30 |
| S733 | Ac-SLEEEWAQIQCEVWGRGCPSESFYGWFERQLG | 31 |
| S767 | Ac-SLEEEWAQIQCEVWGRPCPSESFYGWFERQLG | 32 |
| S873 | Ac-SLEEEW-Aib-QIQCEVWGRPCPSEP-Dip- | 33 |
| | YGWFHEQLGPP | |

TABLE 2
Abbreviations Used in Describing IRBPs Herein and In The PPDs

| | |
|---|---|
| Sar | sarcosine = N-methylglycine |
| Aib | aminoisobutyric acid |
| Dip | diphenylalanine |
| N-MePhe | N-methyl-phenylalanine |
| r | D-arginine |
| Orn | ornithine |
| Tbp | 4-tertbutyl-phenylalanine |
| Pya2 | pyridylalanine |
| Phg | phenylglycine |
| Hph | homophenylalanine |
| Cha | cyclohexylalanine |
| Bip | 4-biphenylalanine |
| Aic | 2-aminoindane-2-carboxylic acid |
| pox | N-Fmoc-8-amino-3,6-dioxaoctanoic acid |
| atan | N-Fmoc-19-amino-5-oxo-3,10,13,16- |
| | tetraoxa-6-aza-nonadecanoic acid |
| Ac | acetyl |
| C14 | C14-monocarboxylic acid |
| HOOC—C19/ | C20-dicarboxylic acid |
| C19-COOH | |
| MeO-PEG5000 | polyethylene glycol, MW = 5000 |
| ε-dde | 1-(4,4-dimethyl-2,6- |
| | dioxocyclohexylidene)ethyl |

### 5. IRBAAS Variants and IRBP Fusion Proteins

Various methods of the invention may be practiced with IRBPs comprising one or more IRBAASs that are highly similar (exhibit high levels of sequence identity to) one or more of the IRBAASs described herein or in the PPDs, but that differ from one or more of the explicitly described sequences by one or more (e.g., about 5 or less, about 3 or less, etc.) acceptable amino acid residue substitutions, additions, or deletions and which bind to IR with the at least substantially similar affinity and/or activate one or more IR activities (e.g., blood glucose lowering) with at least substantially similar activity as the “parent” IRBP. Such IRBMSs can be described as “variants.”

In another aspect, various inventive methods provided herein can be practiced with fusion proteins comprising one or more IRBAASs and/or IRBAAS variants as well as one or more functional fusion partner sequences that impart additional functions and/or physiochemical properties to the IRBP fusion protein that are not present in a “parent” peptide, which lacks the fusion partner sequence(s).

Exemplary fusion partner sequences that can be included in IRBP fusion proteins that can be used in various inventive methods include sequence tags (e.g., FLAG® tags) or purification-enhancing amino acids/sequences, such as one or more lysines, can be added to the peptide sequences of the invention (e.g., at the N-terminal or C-terminal ends). Sequence tags can be used for peptide purification or localization. Lysines can be added to a sequence to increase peptide solubility or to allow for biotinylation. Alternatively, amino acid residues located at the carboxy and amino terminal regions of IRBAASs that comprise sequence tags (e.g., FLAG® tags), or which contain amino acid residues that are not associated with a strong preference for a particular amino acid, may optionally be deleted providing for truncated sequences. Detailed examples of such molecules are described in the PPDs.

Variants also can include amino acid sequences in which one or more residues are modified (e.g., by phosphorylation, sulfation, acylation, PEGylation, etc.). Amino acid sequences may also be modified with a label capable of providing a detectable signal, either directly or indirectly, including, but not limited to, radioisotope, fluorescent, and enzyme labels. Fluorescent labels include, for example, Cy3, Cy5, Alexa, BODIPY, fluorescein (e.g., Fluor X, DTAF, and FITC), rhodamine (e.g., TRITC), auramine, Texas Red, AMCA blue, and Lucifer Yellow. Preferred isotope labels include ³H, ¹⁴C, ³²P, ³⁵S, ³⁵Cl, ⁵¹Cr, ⁵⁷Co, ⁵⁸Co, ⁵⁹Fe, ⁹⁰Y, ¹²⁵I, ¹³¹I, and ¹⁸⁶Re. Typical enzyme labels include peroxidase, β-glucuronidase, β-D-glucosidase, β-D-galactosidase, urease, glucose oxidase plus peroxidase, and alkaline phosphatase (see, e.g., U.S. Pat. Nos. 3,654,090; 3,850,752 and 4,016,043). Enzymes can be conjugated by reaction with bridging molecules such as carbodiimides, diisocyanates, glutaraldehyde, and the like. Enzyme labels can be detected visually, or measured by calorimetric, spectrophotometric, fluorospectrophotometric, amperometric, or gasometric techniques. Other labeling systems, such as avidin/biotin, Tyramide Signal Amplification (TSA™), are known in the art, and are commercially available (see, e.g., ABC kit, Vector Laboratories, Inc., Burlingame, Calif.; NEN® Life Science Products, Inc., Boston, Mass.).

Inventive methods provided here may be practiced in certain aspects with IRBPs that comprise one or more variant IRBAASs (i.e., IRBMSs that differ from one or more parent IR-BMSs specifically disclosed herein, in the PPDs, and/or both (e.g., in the context of a sequence disclosed in the prior patent documents but modified by another principle described herein such as by N-terminal acetylation (or other acylation) and/or C-terminal amidation and/or by inclusion of degradation-resistant unusual amino acid residues and/or non-M moieties) by the relative insertion, deletion, addition, or substitution of one or more amino acid residues). Typically, such IRBP variants comprise one or more IRBMSs exhibit at least about 60%, at least about 70%, at least about 80%, at least about 85%, at least about 90%, about 95%, or more identity (but typically less than 100% identity) to such parent IRBAASs. Typically, variants differ from “parent” IRBAASs mostly through conservative substitutions; e.g., at least about 35%, about 50% or more, about 60% or more, about 70% or more, about 75% or more, about 80% or more, about 85% or more, about 90% or more, about 95% or more (e.g., about 65-99%) of the substitutions in the variant sequence are conservative amino acid residue replacements. In the context of this invention, conservative substitutions can be defined by substitutions within the classes of amino acids reflected in the following table:

TABLE 3
Amino Acid Residue Groupings

| | |
|---|---|
| Alcohol group-containing | S and T |
| residues | |
| Aliphatic residues | I, L, V, and M |
| “Aromatic” residues | F, H, W, and Y |
| Hydrophobic residues | A, C, F, G, H, I, L, M, R, T, V, W, |
| | and Y |
| Negatively charged residues | D and E |
| Polar residues | C, D, E, H, K, N, Q, R, S, and T |
| Positively charged residues | H, K, and R |
| Small residues | A, C, D, G, N, P, S, T, and V |
| Very small residues | A, G, and S |
| Residues involved in turn | A, C, D, E, G, H, K, N, Q, R, S, |
| formation | P, and T |
| Flexible residues | E, Q, T, K, S, G, P, D, E, and R |

Substantial changes in function can be made by selecting substitutions that are less conservative than those shown in the defined groups, above. For example, non-conservative substitutions can be made which more significantly affect the structure of the peptide in the area of the alteration, for example, the alpha-helical, or beta-sheet structure; the charge or hydrophobicity of the molecule at the target site; or the bulk of the side chain. The substitutions which generally are expected to produce the greatest changes in the peptide's properties are those where 1) a hydrophilic residue, e.g., seryl or threonyl, is substituted for (or by) a hydrophobic residue, e.g., leucyl, isoleucyl, phenylalanyl, valyl, or alanyl; 2) a cysteine or proline is substituted for (or by) any other residue; 3) a residue having an electropositive side chain, e.g., lysyl, arginyl, or histidyl, is substituted for (or by) an electronegative residue, e.g., glutamyl or aspartyl; or 4) a residue having a bulky side chain, e.g., phenylalanine, is substituted for (or by) a residue that does not have a side chain, e.g., glycine. Accordingly, these and other nonconservative substitutions can be introduced into peptide variants where significant changes in function/structure is desired and such changes avoided where conservation of structure/function is desired.

Those skilled in the art will be aware of additional principles useful in the design and selection of peptide variants. For example, residues in surface positions of a peptide typically a strong preference for hydrophilic amino acids. Steric properties of amino acids can greatly affect the local structures that a protein adopts or favors. Proline, for example, exhibits reduced torsional freedom that can lead to the conformation of the peptide backbone being locked in a turn and with the loss of hydrogen bonding, often further resulting in the residue appearing on a surface loop of a protein. In contrast to Pro, Gly has complete torsional freedom about a main peptide chain, such that it is often associated with tight turns and regions buried in the interior of the protein (e.g., hydrophobic pockets). The features of such residues often limit their involvement in secondary structures. However, residues typically involved in the formation of secondary structures are known. For example, residues such as Ala, Leu, and Glu (amino acids without much bulk and/or polar residues) typically are associated with alpha-helix formation, whereas residues such as Val, Ile, Ser, Asp, and Asn can disrupt alpha helix formation. Residues with propensity for beta-sheet structure formation/inclusion include Val and Ile and residues associated with turn structures include Pro, Asp, and Gly. The skilled artisan can consider these and similar known amino acid properties in the design and selection of suitable peptide variants, such that suitable variants can be prepared with only routine experimentation.

Desirably, conservation in terms of hydropathic/hydrophilic properties also is substantially retained in a variant peptide as compared to a parent peptide (e.g., the weight class, hydropathic score, or both of the sequences are at least about 50%, at least about 60%, at least about 70%, at least about 75%, at least about 80%, at least about 85%, at least about 90%, at least about 95%, or more (e.g., about 65-99%) retained). Methods for assessing the conservation of the hydropathic character of residues/sequences are known in the art and incorporated in available software packages, such as the GREASE program available through the SDSC Biology Workbench (see also, e.g., Kyte and Doolittle et al., J. Mol. Biol. 157:105-132 (1982); Pearson and Lipman, PNAS (1988) 85:2444-2448, and Pearson (1990) Methods in Enzymology 183:63-98 for a discussion of the principles incorporated in GREASE and similar programs).

It also is typically advantageous that structure of a variant peptide or sequence is substantially similar to the structure of the parent peptide or sequence. Methods for assessing similarity of peptides in terms of conservative substitutions, hydropathic properties, weight conservation, and similar considerations are described in e.g., International Patent Applications WO 03/048185, WO 03/070747, and WO 03/027246. Secondary structure comparisons can be made using the EBI SSM program (currently available at http://www.ebi.ac.uk/msd-srv/ssm/). Where coordinates of the variant are known they can be compared by way of alignment/comparison programs such as DALI pair alignment (currently available at http://www.ebi.ac.uk/dali/Interactive.html), TOPSCAN (currently available at http://www.bioinf.org.uk/topscan), COMPARER (currently available at http://www-cryst.bioc.cam.ac.uk/COMPARER/) PRIDE pair (currently available at http://hydra.iogeb.trieste.it/pride/pride.php?method=pair), PINTS (currently available at http://www.russell.embl.de/pints/), SARF2 (currently available at http://123d.ncifcrf.gov/run2.html), the Structural Alignment Server (currently available at http://www.molmovdb.org/align/), and the CE Calculate Two Chains Server (currently available at http://cl.sdsc.edu/ce/ce_align.html). Ab initio protein structure prediction methods can be applied, if needed, to a variant sequence, such as through the HMM-ROSETTA or MOD-ELLER programs, to predict the structure for comparison with the parent sequence(s) molecule. Where appropriate other structure prediction methods, such as threading methods, also or alternatively can be used, to predict the structure of the variant and/or parent sequence proteins.

The retention of similar residues also or alternatively can be measured by a similarity score, as determined by use of a BLAST program (e.g., BLAST 2.2.8 presently available through the US NCBI). Suitable variants typically exhibit at least about 45%, such as at least about 55%, at least about 65%, at least about 75%, at least about 85%, at least about 90%, at least about 95%, or more (e.g., about 70-99%) similarity to the parent peptide.

As discussed elsewhere herein, other points of variation/divergence between a variant and a parent can be acceptable (e.g., inclusion of non-naturally-occurring amino acids, derivatized amino acids, insertions, deletions, and extensions to the sequence, etc.) provided that such changes do not substantially impair the ability of the variant to bind IR as compared to the parent IRBMS (s).

Identity in the context of amino acid sequences of the invention can be determined by any suitable technique, typically by a Needleman-Wunsch alignment analysis (see Needleman and Wunsch, J. Mol. Biol. (1970) 48:443-453), such as is provided via analysis with ALIGN 2.0 using the BLOSUM50 scoring matrix with an initial gap penalty of −12 and an extension penalty of −2 (see Myers and Miller, CABIOS (1989) 4:11-17 for discussion of the global alignment techniques incorporated in the ALIGN program). A copy of the ALIGN 2.0 program is available, e.g., through the San Diego Supercomputer (SDSC) Biology Workbench. Because Needleman-Wunsch alignment provides an overall or global identity measurement between two sequences, it should be recognized that target sequences which may be portions or subsequences of larger peptide sequences may be used in a manner analogous to complete sequences or, alternatively, local alignment values can be used to assess relationships between subsequences, as determined by, e.g., a Smith-Waterman alignment (J. Mol. Biol. (1981) 147:195-197), which can be obtained through available programs (other local alignment methods that may be suitable for analyzing identity include programs that apply heuristic local alignment algorithms such as FastA and BLAST programs). Further related methods for assessing identity are described in, e.g., International Patent Application WO 03/048185. The Gotoh algorithm, which seeks to improve upon the Needleman-Wunsch algorithm, alternatively can be used for global sequence alignments. See, e.g., Gotoh, J. Mol. Biol. 162:705-708 (1982).

Typically, advantageous sequence changes are those that (1) reduce susceptibility to proteolysis, (2) reduce susceptibility to oxidation, (3) alter binding affinity of the variant sequence (typically desirably increasing affinity), and/or (4) confer or modify other physicochemical or functional properties on the associated variant/analog peptide.

Amino acid sequence variations can result in an altered glycosylation pattern in the variant IRBAAS with respect to a parent IRBMS. “Altering” in this context means removal of one or more glycosylation sites found in the parent IRBMS and/or adding one or more glycosylation sites that are not present in the parent IRBAAS. Glycosylation is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are common recognition sequences for enzymatic attachment of the carbohydrate moiety to the asparagine side chain. Thus, the presence of either of these tripeptide sequences in a polypeptide can create a potential glycosylation site. O-linked glycosylation refers to the attachment of sugars such as N-aceylgalactosamine, galactose, or xylose to a hydroxyamino acid, most commonly serine or threonine, although 5-hydroxyproline or 5-hydroxylysine may also be used. Addition of glycosylation sites to a IRBMS can be conveniently accomplished by altering the amino acid sequence of a variant IRBAAS with respect to the parent sequence such that it is caused to contain one or more of the above-described tripeptide sequences (for N-linked glycosylation sites) or other suitable glycosylation site. The alteration may also be made by, for example, the addition of, or substitution by, one or more serine or threonine residues to the sequence of the original IRBAAS (for O-linked glycosylation sites).

Amino acid sequence variants generally can be obtained by, for example, introducing appropriate nucleotide changes into an IRBAAS-encoding nucleic acid sequence (e.g., by site directed mutagenesis), by chemical peptide synthesis, or any other suitable technique. Such variants include, for example, variants differing by deletions from, insertions into, additions to (at either end of the parent sequence), and/or substitutions of, residues within the parent amino acid sequences. Any combination of deletions, insertions, additions, and substitutions can be made to arrive at a desired variant, provided that the variant possesses suitable characteristics for practice in the methods of the invention (e.g., retention of at least a substantial proportion of the parent sequences affinity for IR). There are a number of more sophisticated techniques that also are readily available for obtaining variants including directed evolution, mutagenesis techniques, and the like.

Suitable variants can be assessed by screening assays described in the prior patent documents including, e.g., surface plasmon resonance (SPR) affinity analysis (e.g., BIAcore™ SPR analysis); IR autophosphorylation assays (e.g., holoenzyme phosphorylation assays); competition assays (e.g., Time-resolved fluorescence resonance energy transfer (TR-FRET) assays); and substrate phosphorylation assays (e.g., a HIR kinase assay); and intravenous blood glucose testing.

### 6. Additional IRBP Derivatives

IRBP derivatives, which specifically include, but are not limited to, enzyme degradation-resistant derivates, acetylated/amidated derivatives, and other derivatives specifically described elsewhere herein, also may typically be used in various inventive methods described herein.

The term derivative generally refers to a protein in which one or more of the amino acid residues of the peptide have been chemically modified (e.g., by alkylation, acylation, ester formation, amide formation, or other similar type of modification) or covalently associated with one or more heterologous substituents (e.g., a lipophilic substituent, a PEG moiety, a peptide side chain linked by a suitable organic moiety linker, etc.). The second type of derivative can separately be described as a conjugate. Because derivatives can vary significantly from their “naked” protein counterparts, uses of such different types of molecules in various methods often can be considered unique aspects of the invention.

In general, IRBPs can be modified by inclusion of any suitable number of such modified amino acids and/or associations with such conjugated substituents. Suitability in this context general is determined by the ability to at least substantially retain (if not increase) the IR binding and agonist activity associated with the non-derivatized parent IRBP/IRBMS. The inclusion of one or more modified amino acids may be advantageous in, for example, (a) increasing polypeptide serum half-life, (b) reducing polypeptide antigenicity, or (c) increasing polypeptide storage stability. Amino acid (s) may be modified, for example, co-translationally or post-translationally, during recombinant production (e.g., N-linked glycosylation at N-X-S/T motifs during expression in mammalian cells) or by synthetic means. Non-limiting examples of a modified amino acid include a glycosylated amino acid, a sulfated amino acid, a prenlyated (e.g., farnesylated, geranylgeranylated) amino acid, an acetylated amino acid, an acylated amino acid, a PEGylated amino acid, a biotinylated amino acid, a carboxylated amino acid, a phosphorylated amino acid, and the like. References adequate to guide one of skill in the modification of amino acids are replete throughout the literature. Exemplary protocols are found in, e.g., Walker (1998) PROTEIN PROTOCOLS ON CD-ROM (Humana Press, Towata, N.J. USA). Typically, a modified amino acid is selected from a glycosylated amino acid, a PEGylated amino acid, a farnesylated amino acid, an acetylated amino acid, a biotinylated amino acid, an amino acid conjugated to a lipid moiety, and an amino acid conjugated to an organic derivatizing agent.

Appropriate methods provided here also may be amenable to practice with IRBPs that are chemically modified by covalent conjugation to a polymer to increase their circulating half-life, for example. Exemplary polymers and methods to attach such polymers to peptides are illustrated in, e.g., U.S. Pat. Nos. 4,766,106; 4,179,337; 4,495,285; and 4,609,546. Additional illustrative polymers include polyoxyethylated polyols and polyethylene glycol (PEG) moieties (e.g., a IRBP can be conjugated to a PEG with a molecular weight of between about 1,000 and about 40,000, such as between about 2000 and about 20,000, e.g., about 3,000-12,000, and even more particularly about 5,000).

IRBPs that may be used in various described methods may be modified so as to manipulate storage stability, pharmacokinetics, and/or any aspect of the bioactivity of the peptide, such as, e.g., potency, selectivity, and drug interaction. Chemical modification to which the peptides may be subjected includes, without limitation, the conjugation to a peptide of one or more of polyethylene glycol (PEG), monomethoxy-polyethylene glycol, dextran, poly-(N-vinyl pyrrolidone) polyethylene glycol, propylene glycol homopolymers, a polypropylene oxide/ethylene oxide co-polymer, polypropylene glycol, polyoxyethylated polyols (e.g., glycerol) and polyvinyl alcohol, colominic acids or other carbohydrate based polymers, polymers of amino acids, and biotin derivatives. PEG conjugation of proteins at Cys residues is disclosed, e.g., in Goodson, R. J. & Katre, N. V. (1990) Bio/Technology 8, 343 and Kogan, T. P. (1992) Synthetic Comm. 22, 2417.

Other useful modifications include, without limitation, acylation, particularly N-terminal acylation of an IRBAAS (e.g., an N-terminally located Formula 6 or FSL IRBMS) as described above, which may be obtained, e.g., using methods and compositions such as described in, e.g., U.S. Pat. No. 6,251,856, and International Patent Application WO 00/55119.

### 7. IRBAAS/IRBP Biological Functions and Physiochemical Properties

IRBPs useful in practicing various methods of the invention also or alternatively may be characterized on the basis of one or more biological functions and/or physiochemical properties that these molecules exhibit.

As already mentioned, IRBPs and IRBAASs are characterized by binding an IR. Unless otherwise stated, aspects of this invention are described with reference to the human IR. However, it should be understood that IRBPs and IRBAASs provided by this invention also or alternatively may bind to other IRs, such as a mouse IR, rat IR, primate IR, pig IR, dog IR, etc.

IRBPs can be characterized on the basis of their ability to specifically bind to one or both sites of IR. In general, an IRBMS binds to either Site 1 or Site 2 of an IR. However, multivalent IRBPs and, more particularly, multivalent multispecific IRBPs are also provided by the invention. Such IRBPs, which are further described elsewhere herein, generally comprise at least one Site 1-specific IRBMS and at least one Site 2-specific IRBAAS.

IRBPs of the invention typically are capable of activating the insulin signaling pathway, as shown by, e.g., increased in vitro lipogenesis and by decreased glucose levels after intravenous (i.v. or IV) administration to pigs and anaesthetized rats. IRBPs can, for example, can increase in vitro lipogenesis in insulin receptor-bearing adipocytes about 10% as effective as human insulin (or more) (e.g., at least about 15% as effective as human insulin), about 25% as effective as human insulin (or more), about 33% as effective as human insulin (or more), about 50% as effective as human insulin (or more), about 60% as effective as human insulin (or more). IRBPs can dose-dependently increase whole-body glucose disposal, with potency in the same range as normal insulin.

Typically, the IRBPs of the invention are peptides of about 70 amino acids or less in length, such as less than about 60 amino acids in length, such as about 50 amino acids or less in length (e.g., about 30-50 amino acids in length).

Surprisingly, IRBAASs relevant to the IRBPs of this invention do not exhibit significant similarity with the amino acid sequence of insulin over more than a few amino acid residues in any particular region of the respective amino acid sequences thereof. The differences in composition of the IRBAAS comprised in the IRBPs of the invention with respect to insulin are associated with various biological characteristics that further serve to distinguish the IRBPs from insulins.

In one exemplary aspect, inventive methods described here are practiced with one or more IRBPs having improved stability towards mammalian (e.g., human) digestive enzymes, such as pepsin, trypsin, chymotrypsin, elastase, and/or carboxypeptidase A. In particular aspects, inventive methods are characterized by use of one or more IRBPs that have at least about 50-fold greater stability, at least about 100-fold greater stability, at least about 150-fold greater stability, or even at least about 200-fold greater stability to one or more of such proteolytic digestive enzymes relative to the stability exhibited by IRBP S597 (described elsewhere herein) towards one or more of such enzymes. The phrase “50-gold greater stability” means that the relevant enzyme takes 50 times longer to degrade the relevant IRBP at a target site as compared to the time it takes to degrade the control peptide (i.e., S597). In one aspect, the stability is attributed, at least in part, to the presence of one or more unusual amino acids or moieties that promote enzymatic degradation resistance. In this respect, various inventive methods described here can be practiced using an IRBP comprising one or more degradation resistance-promoting unusual amino acid residues and/or organic moiety/group, wherein the presence of the residue(s) and/or group(s) increases the stability with respect to a substantially identical IRBP lacking the residue(s) and/or group(s) with respect to degradation by one or more of such enzymes.

In another exemplary aspect, IRBPs used in particular methods of the invention can be characterized by exhibiting IR phosphorylation levels that are significantly lower than that observed with IR binding by insulin. IRBPs used in various inventive methods provided here also may be associated with a different IR phosphorylation profile than insulin.

a. IRBP IR Affinity

IRBPs used in the methods provided here typically exhibit high affinity for IR (K_{d} in the pM range). More particularly, IRBPs typically have or are expected to have an affinity (K_{d}) for IR of between about 10⁻⁷ to about 10⁻¹⁵ M, such as 10⁻⁸ to about 10⁻¹² M, or more particularly typically about 10⁻¹⁰ to about 10⁻¹² M.

In particular aspects, various methods provided here may be practiced with IRBPs that have an affinity for the human insulin receptor (HIR) that is at least about 10%, about 20%, about 30%, about 40%, about 50% or more, such as about 60% or more, about 70% or more, about 80% or more, about 90% or more, or about 95% or more of the affinity exhibited by human insulin. In another aspect, various inventive methods provided here may be practiced with IRBPs that have an affinity for HIR that is about equal to the affinity exhibited by insulin for the HIR. In still another aspect, inventive methods provided here may be practiced with one or more IRBPs that exhibit greater affinity for the HIR than human insulin. For example, IRBPs provided by the PPDs may exhibit about 110% or more, about 150% or more, about 175% or more, or even about 200% or more affinity for HIR than human insulin.

b. IRBP IR Selectivity/Specificity

Insulin-like growth factor-1 (IGF-1) and insulin competitively cross-react with IGF-1R and IR (see, e.g., L. Schäffer, 1994, Eur. J. Biochem. 221:1127-1132). Yet, despite 45% overall amino acid identity, insulin and IGF-1 bind only weakly to each other's receptor. The affinity of each peptide for the non-cognate receptor is about 3 orders of magnitude lower than that for the cognate receptor (see, e.g., Mynarcik, et al., 1997, J. Biol. Chem. 272:18650-18655). The differences in binding affinities may be partly explained by the differences in amino acids and unique domains which contribute to unique tertiary structures of ligands (Blakesley et al., 1996, Cytokine Growth Factor Rev. 7(2):153-9).

IRBPs used in practicing the various methods provided here typically are significantly more specific for IR than IGF-1R. Typically, the IR/IGF-1R binding affinity ratio exhibited by IRBPs is about 100 or more. In particular aspects, inventive methods provided here are practiced using IRBPs that exhibit a preference for IR over IGF-1R marked by an affinity ratio of at least about 1,000; at least about 5,000; at least about 10,000, or greater. In an even more particular aspect, inventive methods are practiced using IRBPs that exhibit a preference for IR over IGF-1R marked by an affinity ratio of about 10,000 to about 100,000.

IRBPs also or alternatively can be characterized on the basis of their inability to activate IGF-1R. Thus, in one aspect, methods of this invention can be characterized on the basis of using one or more IRBPs that are efficacious at IR activation but have little or no significant activity with respect to IGF-1R.

In yet a further aspect, methods of the invention may be practiced using IRBPs that also or alternatively are selective for the IR of a particular species as compared to other species. Thus, for example, methods of the invention may be practiced with one or more IRBPs that exhibit a significant preference for human IR as compared to other mammalian IRs, such as rat IR and pig IR (e.g., a preference marked by an affinity ratio of at least about 1.1, 1.3, 1.4, 1.5, 1.6, 1.7, 1.8, 1.9, 2.0, 2.1, 2.2, 2.3, 2.4, 2.5, 2.6, 2.7, or higher).

In a further aspect still, IRBPs that are selective for an isoform of a particular mammalian IR over another isoform are used in practicing various methods provided herein. Isoforms of IRs are known to exist in several mammalian species. For example, HIR−11 and HIR+11 refer to the two isoforms of the human insulin receptor, without and with exon 11 respectively (such isoforms are apparently generated by an alternative splicing mechanism). These isoforms are also known as HIR A and HIR B. Various inventive methods can be practiced by employing one or more IRBPs that exhibit a preference for HIR−11 over HIR+11 or that exhibit a preference for HIR+11 over HIR-11. HIR+11 and HIR−11, as well as IR isoforms of other species, are expressed at different levels in different tissues. Accordingly, the inventive methods provided here can be advantageously practiced with IRBPs that preferentially associate with different tissue profiles when administered or otherwise delivered to a particular host, such as a human patient.

Selectivity, specificity, affinity, and avidity are concepts well understood in the art (the use of affinity herein may be considered to encompass avidity with respect to multivalent IRBPs), and several techniques are well known and readily available for assessing these measurements with respect to particular IRBPs (as compared to each other and/or different potential binding partners such as IRs of different species and/or an IR of a species as compared to an IGF-1R of the same or different species). Examples of such methods are described, e.g., in the PPDs.

c. IRBP IR Activating Activity

As already suggested, IRBPs exhibit IR agonist activity. IRBPs may, in addition to other characteristics, be characterized on the basis of their ability to lower blood glucose levels, which may be, for example, reflected by the results of a fat cell lipogenesis assay. IRBPs can in this context and other contexts exhibit at least about 10%, at least about 20%, at least about 30%, at least about 40%, at least about 50%, at least about 60%, or more (e.g., about 70-100%) of the blood glucose lowering abilities of human insulin in human insulin receptor-bearing cells. Various methods of the invention may be advantageously practiced with IRBPs that exhibit such activity.

d. IRBP Stability

Methods of the invention may be practiced with one or more IRBPs that exhibit certain levels of stability with respect to enzymatic degradation. For example, various methods provided here may be practiced with an IRBP that is more resistant to degradation by at least one digestive enzyme (e.g., pepsin, chymotrypsin, both, or other similar enzyme) than insulin and that comprises at least one IRBAAS, which IRBMS comprises at least one unusual and digestive enzyme degradation-resistant amino acid residue or other suitable and enzyme degradation-resistant chemical moiety. In one aspect, the unusual amino acid residue/moiety is selected from sarcosine (N-methylglycine); aminoisobutyric acid; diphenylalanine; N-methyl-phenylalanine; D-arginine; ornithine; 4-tertbutyl-phenylalanine; pyridylalanine; phenylglycine; homophenylalanine; cyclohexylalanine; 4-biphenylalanine; 2-aminoindane-2-carboxylic acid; N-Fmoc-8-amino-3,6-dioxaoctanoic acid; N-Fmoc-19-amino-5-oxo-3,10,13,16-tetraoxa-6-aza-nonadecanoic acid; C14-monocarboxylic acid; C20-dicarboxylic acid; polyethylene glycol (PEG) (e.g., a PEG with a molecular weight (MW) of about 5000); and 1-(4,4-dimethyl-2,6-dioxocyclohexylidene)ethyl. In another aspect, methods of the invention may be practiced using a multivalent IRBP comprising at least two IRBAASs, wherein the IRBP comprises at least one unusual enzymatic degradation resistant amino acid residue or chemical moiety located between the IRBMSs. In yet another aspect, methods of the invention may be put into practice with IRBPs comprising such a degradation-resistant unusual residue or moiety located at a terminus of the IRBP. In a further facet, methods of the invention may be practiced using multivalent IRBP comprising at least two IRBAASs, wherein the IRBP comprises at least two of such degradation-resistant residues or moieties. The two or more residues/moieties can be located in a single IRBAAS or in the two or more IRBMS. IRBS comprising any combination of degradation-resistant moieties and/or residues at (a) the termini of the IRBS, (b) between IRBMSs, and/or (c) in one or more IRBAASs, may be useful in various methods described herein.

e. IRBP Effect on the IRACS Pathway

IRBPs suitable for use in the methods of this invention generally can be characterized by exhibiting less of an upregulating effect on one or more components of the IRACS pathway than an equivalent amount of human insulin. In one exemplary aspect, an IRBP can be characterized as exhibiting less upregulation of a HMG-CoA reductase gene (e.g., human HMGCR), a HMG-CoA synthase 1 gene (e.g., human HMGCS1), and/or mevalonate (diphospho) decarboxylase (e.g., human MVD). In particular aspects, an equivalent amount of human insulin exhibits about 2 fold or greater upregulation of HMGCR, HMGCS1, and/or MVD than is exhibited by the IRBP (e.g., about 2.2 fold or greater, about 2.5 fold or greater, about 2.75 fold or greater, about 3 fold or greater). In a further particular and advantageous aspect, IRBPs can downregulate the expression of one or more components of the IRACS pathway (and accordingly, can be used to actually reduce production of cholesterol). In a particular exemplary aspect, IRBPs can be characterized as causing downregulation of one or more of HMGCR, HMGCS1, and/or MVD as compared to prior to administration of about 1.5 fold or greater (e.g., about 1.75 fold or greater downregulation, about 2 fold or greater downregulation, etc.).

### C. Therapeutic and Prophylactic Regimens

### 1. Delivery and Administration Methods

In general, IRBPs can be delivered by any suitable manner in the context of the inventive methods described herein, such as by expression from a nucleic acid that codes for production of the IRBP in target host cells (e.g., by expression from a IRBP-encoding nucleic acid under the control of an inducible promoter and comprised in a suitable gene transfer vector, such as a targeted and replication-deficient gene transfer vector). Typically, IRBPs are delivered by direct administration of the IRBP or IRBP composition to a recipient host. Thus, IRBPs and IRBP compositions may be administered as pharmaceutical compositions comprising standard carriers known in the art for delivering proteins and peptides and/or delivered by gene therapy. In general and where appropriate, the terms administration and delivery should be construed as providing support for one another herein (e.g., it should generally be recognized that IRBP-encoding nucleic acids can be used to deliver naked IRBPs to target host tissues as an alternative to administration of IRBP proteins), although it also should be recognized that each such method is a unique aspect of the invention with respect to any particular molecule and that some molecules (e.g., conjugated IRBPs comprising degradation-resistant organic moieties) are amenable to only certain forms of delivery/administration. Methods for the administration of proteins, nucleic acids, and related compositions (e.g., vectors and host cells), are well known and, accordingly, only briefly described here.

IRBP compositions, related compositions, and combination compositions can be administered via any suitable route, such as an oral, mucosal, buccal, intranasal, inhalable, intravenous, subcutaneous, intramuscular, parenteral, or topical route. Such proteins may also be administered continuously via a minipump or other suitable device.

An IRBP or other IRBP generally will be administered for as long as the disease condition is present, provided that the protein causes the condition to stop worsening or to improve. The IRBP will generally be administered as part of a pharmaceutically acceptable composition, e.g., as described in detail elsewhere herein.

An IRBP may also be administered or otherwise delivered prophylactically to prevent a disease, disorder, or condition for which such treatment may be effective. For example, IRBPs can be administered or otherwise delivered to a patient in remission from a serious diabetic condition (e.g., a significant risk of the onset of diabetes-associated blindness, amputation, or other condition, etc.) in order to reduce the risk of the risk of recurrence diabetes-associated condition.

In general, an IRBP (or related composition such as a vector comprising a IRBP-encoding nucleic acid) may be administered by any suitable route, but typically is administered parenterally in dosage unit formulations containing conventional pharmaceutically acceptable carriers, adjuvants, and the like (stabilizers, disintegrating agents, anti-oxidants, etc.). The term “parenteral” as used herein includes, subcutaneous, intravenous, intraarterial, intramuscular, intrasternal, intratendinous, intraspinal, intracranial, intrathoracic, infusion techniques and intraperitoneal delivery. Thus, in one aspect, an IRBP composition is administered intravenously or subcutaneously, in practicing therapeutic methods of the invention. Routes of injection also include injection into the muscle (intramuscular IM); injection under the skin (subcutaneous (s.c.)); injection into a vein (intravenous (IV)); injection into the abdominal cavity (intraperitoneal (IP)); and other delivery into/through the skin (intradermal delivery, usually by multiple injections, which may include biolistic injections).

In one aspect the invention provides a method of modulating IR activity in a host comprising administering a pharmaceutical composition that includes, in admixture, a pharmaceutically (i.e., physiologically) acceptable carrier, excipient, or diluent, and one or more IR agonist IRBPs as an active agent component (which may be further combined with secondary active agents as described elsewhere).

The pharmaceutical compositions of the invention can be administered systemically by oral or parenteral routes. Non-limiting parenteral routes of administration include subcutaneous, intramuscular, intraperitoneal, intravenous, transdermal, inhalation, intranasal, intra-arterial, intrathecal, enteral, sublingual, or rectal. Due to the labile nature of typical amino acid sequences parenteral administration may be advantageous. Advantageous modes of administration include, e.g., aerosols for nasal or bronchial absorption; suspensions for intravenous, intramuscular, intrasternal or subcutaneous, injection; and compounds for oral administration.

Intravenous administration, for example, can be performed by injection of a unit dose. The term “unit dose” when used in reference to a pharmaceutical composition of the present invention refers to physically discrete units suitable as unitary dosage for humans, each unit containing a predetermined quantity of active material calculated to produce the desired therapeutic effect in association with the required diluent; i.e., liquid used to dilute a concentrated or pure substance (either liquid or solid), making that substance the correct (diluted) concentration for use. For injectable administration, the composition is in sterile solution or suspension or may be emulsified in pharmaceutically- and physiologically-acceptable aqueous or oleaginous vehicles, which may contain preservatives, stabilizers, and material for rendering the solution or suspension isotonic with body fluids (i.e., blood) of the recipient.

Excipients suitable for use are water, phosphate buffered saline, aqueous sodium chloride solution, dextrose, glycerol, dilute ethanol, and the like, and mixtures thereof. Illustrative stabilizers are polyethylene glycol, proteins, saccharides, amino acids, inorganic acids, and organic acids, which may be used either on their own or as admixtures. The amounts or quantities, as well as routes of administration, used are determined on an individual basis, and correspond to the amounts used in similar types of applications or indications known to those of skill in the art.

Pharmaceutical compositions can typically be administered in a manner compatible with the dosage formulation, and in a therapeutically effective amount. The quantity to be administered depends on the subject to be treated, capacity of the subject's immune system to utilize the active ingredient, and degree and type of modulation of IR desired. Precise amounts of active ingredient required to be administered depend on the judgment of the practitioner and are specific for each individual. However, suitable dosages may range from about 10 to 200 nmol active peptide per kilogram body weight of individual per day and depend on the route of administration. Suitable regimes for initial administration and booster shots are also variable, but are typified by an initial administration followed by repeated doses at one or more hour intervals by a subsequent injection or other administration. Alternatively, continuous intravenous infusions sufficient to maintain picomolar concentrations (e.g., approximately 1 pM to approximately 10 nM) in the blood are contemplated. An exemplary formulation comprises an IR agonist IRBP in a mixture with sodium busulfite USP (about 3 mg/ml); disodium edetate USP (about 0.1 mg/ml); and water for injection q.s.a.d. (about 1 ml).

In another particular aspect, an IRBP or an IRBP composition is delivered by an injectable pump in a liquid or other suitable formulation for use with such devices. IRBPs also can be administered by delivery pens, such as are currently used to deliver insulin products. The use of transdermal patches (e.g., a drug in matrix patch) also can be used to deliver IRBPs (e.g., by passive delivery or via iontophoretic delivery).

Further guidance in preparing pharmaceutical formulations can be found in, e.g., Gilman et al. (eds), 1990, Goodman and Gilman's: The Pharmacological Basis of Therapeutics, 8th ed., Pergamon Press; and Remington's Pharmaceutical Sciences, 17th ed., 1990, Mack Publishing Co., Easton, Pa.; Avis et al. (eds), 1993, Pharmaceutical Dosage Forms: Parenteral Medications, Dekker, New York; Lieberman et al. (eds), 1990, Pharmaceutical Dosage Forms: Disperse Systems, Dekker, New York.

a. Exemplary Dosages and Administration Strategies

As described above, compositions of the invention may include a “therapeutically effective amount” or a “prophylactically effective amount” of a IRBP (or first and second amounts in the case of a combination composition comprising a IRBP and a second component; first, second, and third amounts in the case of a combination composition comprising two IRBPs and a secondary agent or a IRBP and two secondary agents; etc.). To better illustrate particular aspects, a detailed discussion of dosage principles is further provided here.

In practicing the invention, the amount or dosage range of the IRBP employed typically is one that effectively induces, promotes, or enhances a physiological response associated with IRBP binding of a cognate IR. In one aspect, the dosage range is selected such that the IRBP employed induces, promotes, or enhances a medially significant effect in a patient suffering from or being at substantial risk of developing a condition associated that is at least in part modulated by IR activity, such as, e.g., a form of diabetes, which effect is associated with the activation, signaling, and/or biological modification (e.g., phosphorylation) of the cognate IR.

In still another aspect, a daily dosage of active ingredient (e.g., IRBP) of about 0.01 to 100 milligrams per kilogram of body weight is provided to a patient. Ordinarily, about 1 to about 5 or about 1 to about 10 milligrams per kilogram per day given in divided doses of about 1 to about 6 times a day or in sustained release form may be effective to obtain desired results.

As a non-limiting example, treatment of IR-associated pathologies in humans or animals can be provided by administration of a daily dosage of IRBP(s) in an amount of about 0.1-100 mg/kg, such as 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day, on at least one of day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively, at least one of week 1, 2, 3, 4, 5, 6, 7, 8, 9, 10, 11, 12, 13, 14, 15, 16, 17, 18, 19 or 20, or any combination thereof, using single or divided doses of every about 24, 12, 8, 6, 4, or 2 hours, or any combination thereof.

In one aspect, the inventive methods comprise administering or otherwise delivering two different IRBPs over a period of one month, the beginning of the therapy involving the second IRBP starting about 1-3 weeks (e.g., about 10 days) after the first delivery of the first IRBP or at any time when a significant immune response to the first IRBP develops in the host, such that the continued use of the first IRBP has become detrimental to the patient.

### 2. Combination Methods: Coadministration and Coapplication

Various combinations of methodologies and/or additional active agents (secondary agents) can be used in practicing the inventive methods described here.

In combination administration/delivery methods, the dose and route of delivery of each of the IRBP and secondary agent(s) can be any suitable dosage and route for achieving the desired therapeutic, prophylactic, and/or physiological effects in the recipient host (e.g., lowering of blood glucose associated with IR activity modulation in a patient). In view of the combined effects of the IRBP and secondary agent in such methods and compositions, the dosage of the IRBP typically is lowered in such methods and compositions with respect to compositions wherein the IRBP is administered alone.

In general, combination administration methods of the invention can comprise any suitable administration scheme, including coadministration (as separate compositions or a single composition wherein the ingredients are mixed or separated) or stepwise administration of the various active agents.

The terms “coadministration,” “coadminister,” and the like herein refer to both to simultaneous administration (or concurrent administration) and serial but related administration, unless otherwise indicated. Coadministration of agents can be accomplished in any suitable manner and in any suitable time. In other words, coadministration can refer to administration of a IRBP before, simultaneously with, or after, the administration of a secondary agent, at any time(s) that result(s) in an enhancement in the therapeutic response over the administration of solely the secondary agent, IRBP, or both agents independently.

Treatment and or prophylactic regiments also can include coapplication of various methods in association with administration or deliver of an IRBP or IRBP composition (e.g., a combination composition as described herein), which may include, for example, application of a low glucose and/or low fat diet; application of an exercise regimen; application of an anti-diabetes gene therapy regimen; application of stem cell or other whole cell therapies (e.g., delivery of insulin-producing β cells—such as ex vivo engineered p cells); application of organ (e.g., pancreas) transplant; transplants of islets; provision of an integrated or connected insulin pump; etc.

When one or more agents are used in combination with an IRBP composition in a therapeutic regimen, there is no requirement for the combined results to be additive of the effects observed when each treatment is conducted separately. Although at least additive effects are generally desirable, any increased IR-mediated effect (e.g., anti-diabetes effect) above one of the single therapies would be of benefit. Also, there is no particular requirement for the combined treatment to exhibit synergistic effects, although this is certainly possible and typically advantageous.

a. Combinations with Anti-Diabetic Agents and Therapies

To practice combined anti-diabetes therapy, effective amounts of an IRBP and secondary anti-diabetes agent (e.g., GLP-1, a GLP-1 analog, a biguanide antidiabetic agent, a glucagon receptor antagonist, etc.) are delivered to a subject or an effective amount of an IRBP is delivered to a patient in coordination with the application of a relevant therapeutic method (e.g., application of a diet therapy) in a manner effective to result in a combined anti-diabetes effect (e.g., reduction of one or more diabetes-associated symptoms and/or physiological conditions). The IRBP and secondary agent or IRBP and method are typically provided or applied in amounts effective and for periods of time effective to result in a combined effect against the disease, disorder or condition. To achieve this goal, an IRBP composition and secondary agents/method may be administered or applied to the animal simultaneously, either in a single combined composition/method, or as two distinct compositions/methods using different administration routes (in the case of combination therapies).

In one aspect, one or more IRBPs are delivered to a patient that is diabetic or pre-diabetic in connection with the delivery of an insulin analogue, typically a long acting insulin analogue, such as, e.g., LysB29(ε-myristoyl)des(B30) human insulin, LysB29(ε-tetradecanoyl)des(B30) human insulin or B29-Nε-(N-lithocolyl-γ-glutamyl)-des(B30) human insulin, wherein the first and second amounts together are effective for treating the disease and typically where the amount of the insulin analogue is significantly less than what would be administered without the IRBP. As used herein, a long-acting insulin analogue is one that exhibits a protracted profile of action relative to native human insulin, as disclosed, e.g., in U.S. Pat. No. 6,451,970. In another aspect, the invention provides the use of a combination composition comprising a therapeutically effective combination of at least one IRBP and at least one insulin or insulin analog in the manufacture of a medicament used in the treatment of disease in a subject, such as in the treatment of type 1 or type 2 diabetes in a subject. Similar compositions comprising combinations of one or more IRBPs and one or more long and/or short-acting insulin analogs also can be suitable for therapeutic methods, such as the treatment of diabetes.

Other antidiabetic agents that can be delivered in connection with IRBPs include insulin, insulin analogues (e.g., Humalog®, NovoLog®, Lantus®, etc.), insulin derivatives, glucagon-like peptide-1 or -2 (GLP-1, GLP-2), derivatives or analogues of GLP-1 or GLP-2 (such as are disclosed, e.g., in WO 00/55119). It will be understood that an “analogue” of insulin, GLP-1, or GLP-2 as used herein refers to a peptide containing one or more amino acid substitutions relative to the native sequence of insulin, GLP-1, or GLP-2, as applicable; and “derivative” of insulin, GLP-1, or GLP-2 as used herein refers to a native or analogue insulin, GLP-1, or GLP-2 peptide that has undergone one or more additional chemical modifications of the amino acid sequence, in particular relative to the natural sequence. Insulin derivatives and analogues are disclosed, e.g., in U.S. Pat. Nos. 5,656,722, 5,750,497, 6,251,856, and 6,268,335. In an exemplary aspect, the secondary antidiabetic agent is selected from LysB29(ε-myristoyl)des(B30) human insulin, LysB29(ε-tetradecanoyl)des(B30) human insulin and B29-Nε-(N-lithocolyl-γ-glutamyl)-des(B30) human insulin. Non-peptide antihyperglycemic agents, antihyperlipidemic agents, and the like, such as those well-known in the art, also may be suitable for combination methods.

In one exemplary aspect, an IRBP or IRBP composition is administered to a patient in association with application of an islet generation method, such as the administration/delivery of an islet-generating molecule, such as an islet-generating C-lectin protein, e.g., Islet Neogenesis Associated Protein (INGAP) or Reg (see, e.g., Kobayashi et al., J Biol Chem. 2000; 275:10723-10726 and Rafaeloff et al., J Clin Invest. 1997; 99:2100-2109).

Additional examples of antidiabetic secondary agents include sulfonaureas, (glipizide (Glucotrol), glimepiride (Amaryl), glyburide, etc., etc.), meglitinides (repaglinide (Prandin) and nateglinide (Starlix)), other insulin secretagogues, biguanides (such as Metformin (Glucophage)), α-Glucosidase inhibitors (e.g., acarbose (Precose) and miglitol (Glyset)), thiazolidinediones (TZDs) (e.g., rosiglitazone (Avandia: GlaxoSmithKline) and pioglitazone (Ac-tos: Eli Lilly and Co.) and other agonists of the peroxisome proliferator-activated receptor-γ (PPARγ), GLP-1 receptor (GLP-1R) agonists (e.g., Exenatide and liraglutide), and/or DPP IV inhibitors (e.g., NVPDPP728 and LAF237).

b. Anti-HCC Combinations

IRBPs also or alternatively can be administered in combination with anti-HCC/anti-HHDRF secondary agents or therapies. Examples of anti-cholesterol agents include resins (Questran and Colestid), triglyceride-lowering drugs (Lopid, Tricor and Niacin), and Statins (Lescol®, Mevacor®, Zocor®, Pravachol®, Lipitor®, and Baycol®). Further classes and types of possible anti-HCC/anti-HHDRF secondary agents include fibric acid derivatives (fibrates), nicotinic acid compounds, bile acid sequestrants, etc. Another particular secondary agent is gemfibrozil. Therapeutically effective amounts of such compounds are known (e.g., doses of about 20-40 mg/d lovastatin, about 40 mg/d pravastatin, about 40 mg/d simvastatin, and about 10 mg/d atorvastatin may be (individually) effective). In one aspect, the amount of anti-HCC/anti-HHDRF used is less than would be used in connection with insulin or an insulin analog.

In patients with low levels of both LDL and HDL cholesterol, gemfibrozil, 1200 mg/d, has also shown benefit.

c. Other Combination Therapies and Methods

Other potentially useful combinations and combination therapies include therapies that upregulate the ABC (HDL production) gene and/or that downregulate the expression of the MTP gene (so as to lower LDL production).

Combination methods also may include, e.g., a treatment plan that includes dietary modifications in a patient such as adopting a low glucose, low fat, and/or low glucose and low fat diet) and/or the adoption of a lifestyle that involves increased routine exercise.

### EXPERIMENTAL SECTION

The following discussion of experimental methods are provided to further illustrate particular aspects of the invention but should not be understood as in any way limiting its scope.

### Overview

To compare the transcriptional effects of human insulin (HI) and an exemplary IRBP, IRBP S597, cDNA microarray analyses were performed on total RNA from human SGBS adipocytes treated with S597 (30 nM) or human insulin (30 nM) for 18 hours. The microarray hybridizations were carried out as dual color (Cy3/Cy5) hybridizations comparing vehicle treatments to insulin or S597 treatments. Three individual RNA samples from vehicle, S597, and insulin treated cells were compared, and all hybridizations were repeated with reversed dye combination (e.g. vehicle (Cy3) vs. S597 (Cy5) repeated as vehicle (Cy5) vs. S597 (Cy3)). The specific steps employed in this experimental analysis of the effects of HI versus IRBP S597 are described in the following paragraphs.

### Methods

### SGBS Cell Cultures

A human preadipocyte cell strain derived from subcutaneous adipose tissue of an infant with Simpson-Golabi-Behmel syndrome (SGBS) was plated out in 6 well plates and grown to confluence in DMEM/F12 medium (Gibco) containing 1% biotin, 1% panthothenic acid, 1% penicillin (10000 U)/streptomycin (10000 U), and 10% Fetal bovine serum (Gibco-Invitrogen, Carlsbad Calif. (USA)—“Gibco”). Differentiation of SGBS preadipocytes into adipocytes was induced by adding DMEM/F12 medium (Gibco) containing 1% biotin, 1% panthothenic acid, 1% penicillin (10,000 U)/streptomycin (10,000 U), 100 nM cortisol, 200 μM triiodothyronine, 20 nM insulin, 250 nM dexamethasone (first 6 days), 500 μM IBMX (first 6 days), 2 μM rosiglitazone (first 3 days) and letting cells differentiate for 14 days.

Before stimulation with insulin and IRBP S597 the differentiated adipocytes were cultured in DMEM/F12 medium (Gibco) containing 1% biotin, 1% panthothenic acid, 1% penicillin (10,000 U)/streptomycin (10,000 U), 100 nM cortisol, and 200 μM triiodothyronine for 2 days. Insulin and S597 (1, 3, 10, 30, 100 nM) were separately added to cells in DMEM/F12 medium (Gibco) containing 1% biotin, 1% panthothenic acid, 1% penicillin (10,000 U)/streptomycin (10,000 U), 100 nM cortisol, 200 pM triiodothyronine, and 0.1% BSA. Following stimulation for 18 h (18 hours), the cells were washed twice in PBS and 1.3 ml SV RNA-lysis buffer (Promega, Madison, Wis.) was added to each well.

### Isolation and Culturing of Hepatocytes

Male Wistar rats (˜200 g) were anaesthetized with a freshly prepared mixture (1:1) of Hyp-norn (0.05 mg/ml fentalyl/2.5 mg/ml fluabizone) and Dormicum (1.25 mg/ml midazolam) administered subcutaneously (1 ml/kg). Hepatocytes were isolated from rats fed ad libitum by a two-step perfusion technique essentially as described by Seglen, Biochem. J. (1999) 342 (545-550). Cell viability, assessed by Trypan Blue exclusion, was consistently greater than 80%. Cells were plated on to collagen-coated six-well plates in basal medium (Medium 199, 5.5 mM glucose supplemented 100 nM decadron, 1% penicillin (10,000 U)/streptomycin (10,000 U), and 1 nM insulin) with 4% fetal calf serum at a cell density of 1.2*10⁶ cells/well. The medium was replaced 1 h after initial plating in order to remove dead cells. Insulin and IRBP S597 (1, 3, 10, 30, 100 nM) were added to the cells in Medium 199, 5.5 mM glucose supplemented 100 nM decadron, 1% penicillin (10000 U)/streptomycin (10000 U) and 0.1% BSA. Following stimulation for 18 h, the cells were washed twice in PBS and 1.3 ml SV RNA-lysis buffer (Promega, Madison, Wis.) were added to each well.

### RNA-Preparation

Total RNA was isolated and DNase treated using SV96 total RNA isolation system (Promega, Madison, Wis.) following manufacturer's instructions.

### Quantitative PCR Analyses

cDNA was prepared from 100 ng of total RNA from each of the treatments (n=3) using random primers and TaqMan® Reverse (Applied Biosystems, Foster City, Calif. (USA)).

Transcription reagents were used/applied according to the manufacturer's instructions. Quantitative PCR was performed on each of the cDNA samples (10-fold dilutions of cDNA) using TaqMan® PCR core reagents (Applied Biosystems) on an ABI PRISM® 7000 Sequence Detection System (Applied Biosystems). Primers and FAM-labeled-probes for human and rat HMG-CoA reductase (HMGCR), mevalonate (diphospho) decarboxylase (MVD), HMG-CoA synthase 1 (HMGCS1), 18S rRNA, rat fatty acid synthase (FAS), and rat glucose-6-phosphate catalytic subunit (G6PC) were ordered as Assays-on-Demand (Applied Biosystems).

Probe sequences for these assays were as follows: HMGCR (human AC-CATGTCAGGGGTACGTCAGCTTG (assay Hs00168352_m1) (SEQ ID NO: 34), rat GCAC-CATGTCAGGGGTGCGGCAGCT (assay Rn00565598_m1) (SEQ ID NO: 35)), MVD (human TCAAGTACTGGGGCAAGCGCGATGA (assay Hs00159403_m1) (SEQ ID NO: 36), rat TCAAATACTGGGGAAAGCGGGATGA (assay Rn00579216_m1) (SEQ ID NO: 37)), HMGCS1 (human CMGATGCTACACCGGGGTCTGCTC (assay Hs00266810_m1) (SEQ ID NO: 38), rat TCCTTCACACAGCTCTTTCACCATG (assay Rn00568579_m1) (SEQ ID NO: 39)), 18S rRNA (TGGAGGGCAAGTCTGGTGCCAGCAG; assay HS99999901_s1) (SEQ ID NO: 40), FAS (rat GGAAGGCTGGGCTCTATGGGTTGCC (assay Rn00569117_m1) (SEQ ID NO: 41)), and G6PC (rat ATGGATTCCGGTGCTTGAATGTCGT (assay Rn00565347_m1) (SEQ ID NO: 42)). Data were analyzed using ABI Prism 7000 SDS software (version 1.0; Applied Biosystems), and expression levels for HMGCR, MVD, HMGCS1, FAS, and were normalized to the 18S rRNA levels.

### Microarray Analyses

Incyte Easy-To-Spot™ PCR-products (ETS1220 comprising ˜9000 human clones) (Amersham Biosciences) dissolved in 50% DMSO were spotted on to Corning Ultra Gaps slides (Corning Inc., Corning, N.Y.) at 55% relative humidity using the Molecular Dynamics Gen. III microarray spotter (Amersham Biosciences, Buckinghamshire, UK). All slides were baked at 80° C. for 4 h. Approximately 250 ng of total RNA from cells treated with vehicle, human insulin (30 nM) and S597 (30 nM) were used for synthesis of complementary RNA (amplified RNA (aRNA)) using Amino-allyl MessageAmp™ aRNA kit (Ambion, Austin, Tex.). Amino-allyl cDNA was synthesized from 1.5 μg of each RNA-amplification. The aRNA was incubated with random primer at 70° C. for 10 min. and subsequently chilled on ice for 30 sec. The primer annealed RNA was incubated with reaction mixture (1× Superscript II buffer (Life Technologies, Taastrup, Denmark), 10 mM DTT, 200 μM dGAT(−TP) 100 μM dCTP, 100 μM Cy-3/Cy-5-dCTP (Amersham Biosciences), and 200 units of Superscript II reverse transcriptase (Life Technologies, Taastrup, Denmark) at 42° C. for 2 h in a final volume of 20 μl. The RNA template was removed by alkaline denaturation (incubation with 2 μl 2.5 M NaOH at 37° C. for 15 min.). Amino-allyl labeled probes were purified using a Qiagen PCR purification kit (Qiagen). The amino-allyl cDNA was resuspended in 0.1 M NaHCO3 pH 9.0 and coupled to Cy3/Cy5 NHS-ester (Amersham Biosciences) for 1 h at RT in the dark. Unreacted ester groups were quenched by addition of hydroxylamine. Cy3- and Cy5-labeled cDNA was purified using Qiagen PCR purification kit (Qiagen). Dual color hybridizations were performed by combining 30 pmol Cy3 labeled cDNA (e.g. from vehicle treated cells) and 30 pmol Cy5 labeled cDNA (e.g. from insulin treated cells) in 50% formamide and 25% Microarray hybridization buffer version2 (Amersham Biosciences) and adding the probe solution to a prespotted Corning UltraGaps slide. Slides were hybridized overnight at 42° C. in a humidified chamber and subsequently washed in (1×SSC, 0.2% SDS) at 55° C. for 10 min., (0.1×SSC, 0.2% SDS) at 55° C. for 2×10 min., and finally in (0.1×SSC) at RT for 2×1 min. Slides were scanned using a GenePix 4000B microarray scanner (Axon Instruments, Union City, Calif.).

### Results

### Microarray Analyses

Three genes in the cholesterol biosynthesis pathway that were upregulated by human insulin but not upregulated or downregulated by S597 were identified by the above-described analysis. Specifically, the microarray analysis described above revealed that genes encoding HMG-CoA reductase (HMGCR), HMG-CoA synthase 1 (HMGCS1), and mevalonate (diphospho) decarboxylase (MVD) were all upregulated by insulin (HMGCR: 2.3 fold, HMGCS1: 2.0 fold, MVD: 2.1 fold) but downregulated by S597 (HMGCR: 2.1 fold, HMGCS1: 1.4 fold, MVD: 2.5 fold).

### Quantitative PCR Analyses

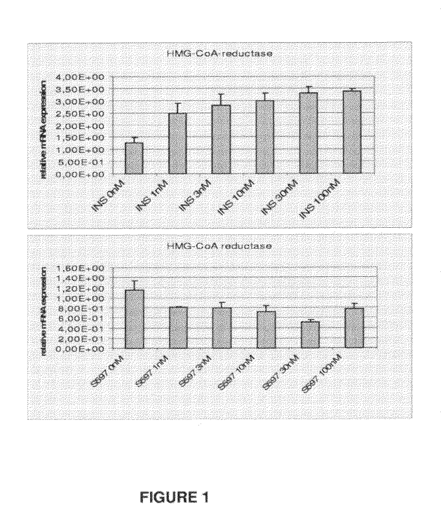

To confirm the identified regulations of HMGCR, HMGCS1, and MVD in SGBS cells treated with insulin or S597, quantitative RT-PCR analyses on 5 different doses (1, 3, 10, 30, 100 nM) of insulin and S597 was performed. The quantitative RT-PCR could confirm a dose dependant upregulation of HMG-CoA reductase (˜3 fold up) by insulin and a dose dependant downregulation (˜2 fold down) by S597 in SGBS-adipocytes, as reflected in FIG. 1.

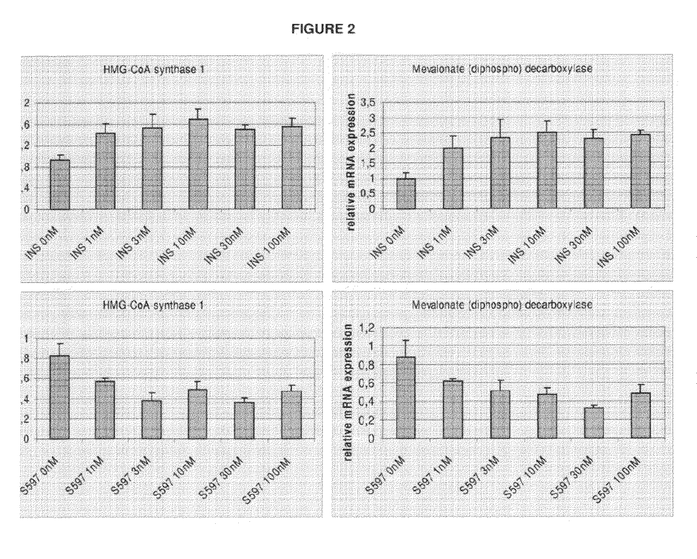

The expression levels of HMG-CoA synthase 1 and mevalonate (diphospho) decarboxylase were likewise upregulated by insulin and downregulated by S597 in a dose dependent manner in the SGBS adipocytes, as reflected in FIG. 2.

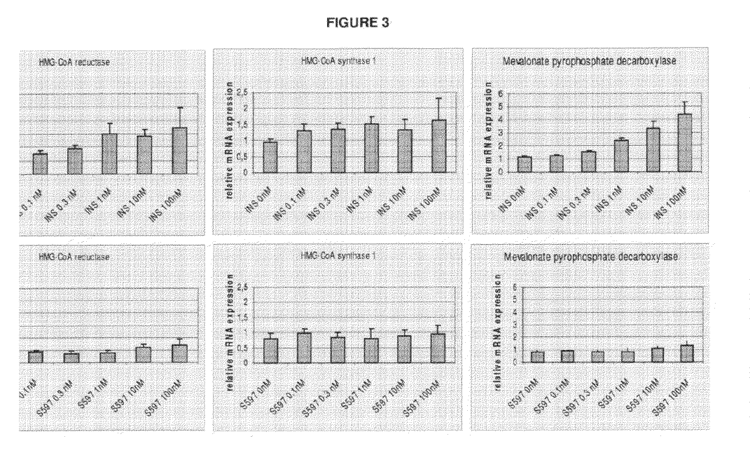

The expression levels of HMGCR, HMGCS1, and MVD were next examined in primary rat hepatocytes treated with 5 different doses of insulin and S597 (0.1, 0.3, 1, 10, 100 nM) for 18 h. As in the SGBS adipocytes, insulin had a clear upregulating effect on the mRNA levels for these genes as reflected in FIG. 3.

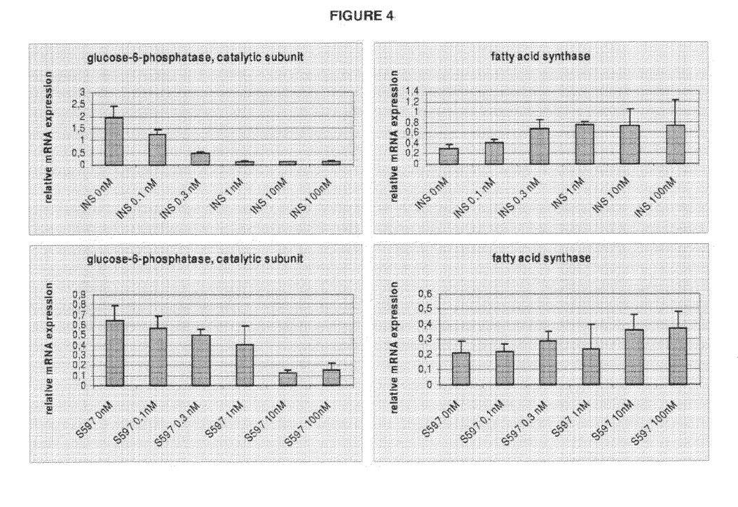

For the S597 treatments, no significant changes were observed. In order to test whether S597 had any stimulatory effects on the primary rat hepatocytes, the mRNA expression of two well known insulin regulated genes, fatty acid synthase and glucose-6-phosphate catalytic subunit, were tested. As shown in FIG. 4, both insulin and S597 exhibited the expected downregulation of glucose-6-phosphate catalytic subunit and upregulation of fatty acid synthase.

Together, these data illustrate a coordinated up-regulation of genes encoding enzymes of the cholesterol biosynthesis pathway by insulin in contrast to S597, which exhibits no stimulatory effects on the mRNA levels of the examined enzymes of cholesterol biosynthesis. It is expected that other IRBPs, particularly Formula 2/Formula 1 and Formula 6/Formula 1 IRBPs will exhibit similar differences in gene activation profiles from human insulin, while still lowering blood glucose levels.

All references, including publications, patent applications, and patents, cited herein are hereby incorporated by reference to the same extent as if each reference were individually and specifically indicated to be incorporated by reference and were set forth in its entirety herein.

All headings and sub-headings are used herein for convenience only and should not be construed as limiting the invention in any way.

Any combination of the above-described elements in all possible variations thereof is encompassed by the invention unless otherwise indicated herein or otherwise clearly contradicted by context.

The use of the terms “a” and “an” and “the” and similar referents in the context of describing the invention are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein. Unless otherwise stated, all exact values provided herein are representative of corresponding approximate values (e.g., all exact exemplary values provided with respect to a particular factor or measurement can be considered to also provide a corresponding approximate measurement, modified by “about,” where appropriate).

All methods described herein can be performed in any suitable order unless otherwise indicated herein or otherwise clearly contradicted by context.

The use of any and all examples, or exemplary language (e.g., “such as”) provided herein, is intended merely to better illuminate the invention and does not pose a limitation on the scope of the invention unless otherwise indicated. No language in the specification should be construed as indicating any element is essential to the practice of the invention unless as much is explicitly stated.

The citation and incorporation of patent documents herein is done for convenience only and does not reflect any view of the validity, patentability, and/or enforceability of such patent documents.

The description herein of any aspect or embodiment of the invention using terms such as “comprising”, “having,” “including,” or “containing” with reference to an element or elements is intended to provide support for a similar aspect or embodiment of the invention that “consists of”, “consists essentially of”, or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context).

This invention includes all modifications and equivalents of the subject matter recited in the aspects presented herein to the maximum extent permitted by applicable law.

1. A method of reducing blood glucose level in a subject having a condition in which upregulation of the insulin receptor (IR)-associated cholesterol synthesis (“IRACS”) pathway is undesirable comprising delivering to the subject a physiologically effective amount of an insulin receptor binding peptide (“IRBP”) so as to reduce blood level therein.

2. The method of claim 1, wherein the subject is a human patient that has diabetes.

3. The method of claim 1, wherein the subject is a human that has pre-diabetes.

4. The method of claim 1, wherein the subject is a human that has at least two high cholesterol condition-associated heart disease risk factors.

5. The method of claim 1, wherein the subject is a human that determined to have a total cholesterol level of more than about 200 mg/dl and/or a total LDL cholesterol level of more than about 100 mg/dl.

6. The method of claim 5, wherein the subject is a human determined to have a total cholesterol level of more than about 230 mg/dl and/or a total LDL cholesterol level of more than about 130 mg/dl.

7. The method of claim 1, wherein the IRBP is delivered by pulmonary administration.

8. The method of claim 1, wherein the IRBP is delivered to the subject by oral administration.

9. The method of claim 2, wherein an approximately equivalent amount of human insulin upregulates expression of HMG-CoA reductase by at least two times the level expressed upon delivery of the IRBP.

10. The method of claim 2, wherein the IRBP is delivered in connection with a secondary anti-diabetic agent and the amounts of the IRBP and the secondary anti-diabetic agent are together effective to reduce blood glucose in the subject.

11. (canceled)

12. The method of claim 2, wherein the subject is a human that has at least two high cholesterol condition-associated heart disease risk factors.

13. The method of claim 3, wherein the subject is a human that has at least two high cholesterol condition-associated heart disease risk factors.

14. The method of claim 2, wherein the subject is a human determined to have a total cholesterol level of more than about 200 mg/dl and/or a total LDL cholesterol level of more than about 100 mg/dl.

15. The method of claim 3, wherein the subject is a human determined to have a total cholesterol level of more than about 200 mg/dl and/or a total LDL cholesterol level of more than about 100 mg/dl.

16. The method of claim 4, wherein the subject is a human determined to have a total cholesterol level of more than about 200 mg/dl and/or a total LDL cholesterol level of more than about 100 mg/dl.

17. The method of claim 3, wherein an approximately equivalent amount of human insulin upregulates expression of HMG-CoA reductase by at least two times the level expressed upon delivery of the IRBP.

18. The method of claim 3, wherein the IRBP is delivered in connection with a secondary anti-diabetic agent and the amounts of the IRBP and the secondary anti-diabetic agent are together effective to reduce blood glucose in the subject.

19. The method of claim 14, wherein the subject is a human determined to have a total cholesterol level of more than about 230 mg/dl and/or a total LDL cholesterol level of more than about 130 mg/dl.

20. The method of claim 15, wherein the subject is a human determined to have a total cholesterol level of more than about 230 mg/dl and/or a total LDL cholesterol level of more than about 130 mg/dl.

21. The method of claim 16, wherein the subject is a human determined to have a total cholesterol level of more than about 230 mg/dl and/or a total LDL cholesterol level of more than about 130 mg/dl.


SEQUENCE LISTING (42 sequences)

SEQ ID NO: 1 - protein, 5 aa, Artificial
  Synthetic
FYXWF

SEQ ID NO: 2 - protein, 5 aa, Artificial
  Synthetic
FYDWF

SEQ ID NO: 3 - protein, 5 aa, Artificial
  Synthetic
FYEWF

SEQ ID NO: 4 - protein, 5 aa, Artificial
  Synthetic
FYGWF

SEQ ID NO: 5 - protein, 9 aa, Artificial
  Synthetic
XYXWXERQL

SEQ ID NO: 6 - protein, 10 aa, Artificial
  Synthetic
XYXWXERQLG

SEQ ID NO: 7 - protein, 10 aa, Artificial
  Synthetic
XYGWXERQXG

SEQ ID NO: 8 - protein, 10 aa, Artificial
  Synthetic
XYXWXERQLG

SEQ ID NO: 9 - protein, 9 aa, Artificial
  Synthetic
FYXWFERQL

SEQ ID NO: 10 - protein, 16 aa, Artificial
  Synthetic
XXXXFYXYFXXLXXXX

SEQ ID NO: 11 - protein, 12 aa, Artificial
  Synthetic
SEGFYNAIELLS

SEQ ID NO: 12 - protein, 20 aa, Artificial
  Synthetic
XLXXXWXXXXCXXXXXXCXX

SEQ ID NO: 13 - protein, 18 aa, Artificial
  Synthetic
XLEXEWXXXXXXVYXXXX

SEQ ID NO: 14 - protein, 18 aa, Artificial
  Synthetic
SLEEEWAQIEXEVWGRGX

SEQ ID NO: 15 - protein, 32 aa, Artificial
  Synthetic
SLEEEWAQIEAEVWGRGAPSESFYDWFERQLG

SEQ ID NO: 16 - protein, 32 aa, Artificial; identical to SEQ ID NO: 15
  Synthetic

SEQ ID NO: 17 - protein, 18 aa, Artificial
  Synthetic
XLEXEWXXXXXXVYXXXX

SEQ ID NO: 18 - protein, 18 aa, Artificial
  Synthetic
SLEEEWAQIXXEVWGRGX

SEQ ID NO: 19 - protein, 18 aa, Artificial
  Synthetic
XLEXEWXXXXXXVYXXXX

SEQ ID NO: 20 - protein, 36 aa, Artificial
  Synthetic
SLEEEWAQVECEVYGRGCPSGSLDESFYDWFERQLG

SEQ ID NO: 21 - protein, 31 aa, Artificial
  Synthetic
SLEEEWAQIECEVYGRGCPSESFYDWFERQL

SEQ ID NO: 22 - protein, 30 aa, Artificial
  Synthetic
SLEEEWAQIECEVWGRGCPKGFYGWFRRRG

SEQ ID NO: 23 - protein, 31 aa, Artificial; identical to SEQ ID NO: 21
  Synthetic

SEQ ID NO: 24 - protein, 33 aa, Artificial
  Synthetic
SLEEEWAQIQCEVWGRGCPSESFYDWFEAQLHA

SEQ ID NO: 25 - protein, 31 aa, Artificial
  Synthetic
SLEEEWAQIQCEVWGRGCPSESFYDWFERQL

SEQ ID NO: 26 - protein, 32 aa, Artificial
  Synthetic
SLEEEWAQIQCEVWGRGCQRPEPFYDWFERQL

SEQ ID NO: 27 - protein, 31 aa, Artificial
  Synthetic
SLEEEWAQIECEVYGRGCPSESFYHWFERQL

SEQ ID NO: 28 - protein, 32 aa, Artificial
  Synthetic
SLEEEWAQIQCEVWGRGCPSESFYDWFERQLG

SEQ ID NO: 29 - protein, 31 aa, Artificial
  Synthetic
SLEEEWAQIECEVWGRGCPSEGFYNAIELLS

SEQ ID NO: 30 - protein, 34 aa, Artificial
  Synthetic
SLEEEWAQIEAEVWGRGAPSESFYDWFERQLGNH

SEQ ID NO: 31 - protein, 32 aa, Artificial
  Synthetic
SLEEEWAQIQCEVWGRGCPSESFYGWFERQLG

SEQ ID NO: 32 - protein, 32 aa, Artificial
  Synthetic
SLEEEWAQIQCEVWGRPCPSESFYGWFERQLG

SEQ ID NO: 33 - protein, 34 aa, Artificial
  Synthetic
SLEEEWXQIQCEVWGRPCPSEPXYGWFHEQLGPP

SEQ ID NO: 34 - DNA, 25 bp, Artificial
  Synthetic
accatgtcaggggtacgtcagcttg

SEQ ID NO: 35 - DNA, 25 bp, Artificial
  Synthetic
gcaccatgtcaggggtgcggcagct

SEQ ID NO: 36 - DNA, 25 bp, Artificial
  Synthetic
tcaagtactggggcaagcgcgatga

SEQ ID NO: 37 - DNA, 25 bp, Artificial
  Synthetic
tcaaatactggggaaagcgggatga

SEQ ID NO: 38 - DNA, 25 bp, Artificial
  Synthetic
caagatgctacaccggggtctgctc

SEQ ID NO: 39 - DNA, 25 bp, Artificial
  Synthetic
tccttcacacagctctttcaccatg

SEQ ID NO: 40 - DNA, 25 bp, Artificial
  Synthetic
tggagggcaagtctggtgccagcag

SEQ ID NO: 41 - DNA, 25 bp, Artificial
  Synthetic
ggaaggctgggctctatgggttgcc

SEQ ID NO: 42 - DNA, 25 bp, Artificial
  Synthetic
atggattccggtgcttgaatgtcgt